(19) United States
(12) Patent Application Publication
(10) Pub. No.: US 2014/0018574 A1
(43) Pub. Date: Jan. 16, 2014

(54) PROCESS FOR PREPARING ACRYLIC ACID BY A THERMOLYSIS OF POLY-3-HYDROXYPROPIONATE CATALYZED BY AT LEAST ONE MOLECULAR ACTIVE COMPOUND

(71) Applicant: BASF SE, Ludwigshafen (DE)

(72) Inventors: Christian RAITH, Mannheim (DE); Marek Pazicky, Heidelberg (DE); Rocco Paciello, Bad Duerkheim (DE); Raphael Heinrich Brand, Rossdorf (DE); Marco Hartmann, Woerth (DE); Klaus Joachim Mueller-Engel, Stutensee (DE); Peter Zurowski, Landau (DE); Wolfgang Fischer, Lingenfeld (DE)

(21) Appl. No.: 13/941,856

(22) Filed: Jul. 15, 2013

Related U.S. Application Data
(60) Provisional application No. 61/671,823, filed on Jul. 16, 2012.

(30) Foreign Application Priority Data
Jul. 16, 2012 (DE) 10 2012 212 424.1

(51) Int. Cl. C07C 51/377

(52) U.S. Cl. CPC C07C 51/377
USPC 562/599

(57) ABSTRACT

A process for preparing acrylic acid by thermolysis of poly-3-hydroxypropionate in the presence of one or more specific tertiary amines as a catalyst.

The present invention relates to a process for preparing acrylic acid by a thermolysis of poly-3-hydroxypropionate catalyzed by at least one molecular organic active compound having at least one tertiary nitrogen atom which has a covalent bond to three different carbon atoms in the molecular organic active compound.

Acrylic acid is an important monomer which, owing to its marked tendency to free-radical polymerization, finds use as such, in the form of its alkyl esters and/or in the form of its alkali metal salts especially for preparation of polymers obtainable by free-radically initiated polymerization.

Depending on the individual acrylic monomers used for formation of the respective polymer, it can be used, for example, as an adhesive or as a superabsorbent for water or aqueous solutions. The latter are polymers in which at least a portion of the polymerized acrylic acid is in a form neutralized with alkali metal bases, for example NaOH (cf., for example, DE 102004004496 A1 and DE 102011076931 A1). These polymers generally have a marked absorption tendency for aqueous liquids (cf., for example, US 2010/0041549 and “Modern Superabsorbent Polymer Technology”, Buchholz/Graham, Wiley VCH, New York, 1998).

The field of use thereof is especially in the sector of hygiene articles, for example diapers, and particularly high demands are therefore made on the purity of acrylic acid used for the preparation thereof.

A disadvantage of acrylic acid is, however, that the capacity thereof for free-radical polymerization is so marked that it frequently sets in not just when it is triggered in an intentional manner by means of suitable free-radical initiators. In other words, acrylic acid, especially in the condensed phase, has a non-negligible tendency to unwanted free-radical polymerization (for example initiated by ever-present thermal energy and/or electromagnetic radiation), and this can take a comparatively violent and uncontrolled course due to the exothermicity thereof.

In the course of storage and/or transport of acrylic acid, it is therefore necessary for safety reasons to counteract such unwanted free-radical polymerization by addition of polymerization inhibitors to the acrylic acid. Such an addition, however, is disadvantageous in that it impairs any later intentionally initiated free-radical polymerization.

A further disadvantage of acrylic acid is due to the fact that, present in the liquid phase, it unavoidably ages with time as a result of Michael addition onto itself and onto the addition products which form.

While acrylic acid thus has an excellent “reaction form”, its “storage form (depot form)/transport form” is not entirely satisfactory.

A significantly more advantageous depot form/transport form in this respect of acrylic acid is poly-3-hydroxypropionate.

In this document, this is understood to mean macromolecular compounds having at least one structural section of the general formula I

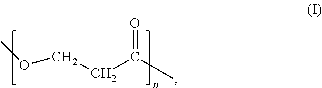

where n is an integer ≧6.

The structural section of the general formula I is the polycondensate (the polyester) of 3-hydroxypropionic acid (=the hydrate of acrylic acid) with itself.

Poly-3-hydroxypropionates, in contrast to acrylic acid, are essentially not subject to any aging process under standard conditions (=25° C. and a pressure of 1.0133·10⁵ Pa (=standard pressure)). More particularly, poly-3-hydroxypropionate, which is typically in the solid state of matter under standard conditions, can be both stored and transported without any problem.

The prior art discloses that structural sections of the general formula (I) present in a poly-3-hydroxypropionate can be split by the sole action of elevated temperatures (of an elevated temperature) as required to give acrylic acid (the dehydrate of 3-hydroxypropionic acid) (cf., for example, U.S. Pat. No. 2,568,636 A, U.S. Pat. No. 2,361,036 A and EP 577206 A2).

The acrylic acid can be converted from the gas phase which comprises acrylic acid and is obtained in the thermal splitting (in the “thermolysis”) to the liquid phase in a manner known per se by absorptive and/or condensative measures. In general, this liquid phase may already be the acrylic acid suitable for further uses, for example free-radical polymerizations. Especially when the acrylic acid thus obtained can be supplied without intermediate storage to the further use thereof in the context of, for example, a free-radically initiated polymerization, it will be possible to undertake the aforementioned conversion of the acrylic acid to the liquid phase advantageously without additional use of polymerization inhibitors (which impair a free-radically initiated polymerization).

A further disadvantage of acrylic acid obtained as described by thermolysis of poly-3-hydroxypropionate (or originating from such a thermolysis) is that it does not have the fingerprint of low molecular weight aldehydes present therein as impurities which is a typical result in the case of preparation processes of acrylic acid by heterogeneously catalyzed partial oxidations of C₃ precursor compounds of acrylic acid (e.g. propylene, propane, acrolein, glycerol, propionic acid, propanol etc.) (cf., for example, DE 102011076931 A1).

Such aldehydes, even in amounts of 1 to 10 ppm by weight, based on the weight of the mass of acrylic acid, are found to be extremely disruptive in the case of use of the acrylic acid and/or of the conjugated (Brønsted) base for preparation of polymers by free-radically initiated polymerization, optionally in a mixture with other mono- and/or polyunsaturated (for example mono- and/or polyethylenically unsaturated) compounds (for example, they can undesirably retard the free-radically initiated polymerization or impair the preparation of polymer with particularly high molecular weight (as is desirable especially in the superabsorbance sector) owing to their “regulating action”).

It is also known from the prior art that the temperatures required for appropriate splitting rates in the case of thermolysis of poly-3-hydroxypropionate to acrylic acid can be considerably reduced by the addition of suitable splitting catalysts to the poly-3-hydroxypropionate to be split (or to a splitting mixture comprising the latter).

As potential splitting catalysts of this kind, WO 2011/100608 A1 considers a comparatively wide variety of chemical substance classes (which in a formal sense also comprise organic amines), but these do not show any unifying structural feature essential for advantageous usability as such a splitting catalyst.

By way of example, the splitting catalysts used in WO 2011/100608 A1 are merely nonvolatile salts such as Na₂CO₃, FeSO₄.7H₂O and Ca(OH)₂.

Use of salts as splitting catalysts, however, is disadvantageous in that they necessarily remain in the splitting residues due to the nonvolatility thereof.

WO 2011/100608 proposes, in this regard, completely decomposing the organic constituents of the splitting residues by corresponding thermal action to leave salts present, in order to be able to reuse the remaining salts as splitting catalysts, but the reusability of such remaining salt residues as splitting catalysts is generally impaired as a result of carbon deposits, for example, present therein and due to chemical change which has occurred (e.g. Na₂CO₃→Na₂O). However, disposal of salt residues is generally costly.

U.S. Pat. No. 2,361,036 considers, as catalysts for a thermolysis of poly-3-hydroxypropionate, those substances which are also considered as catalysts for preparation of poly-3-hydroxypropionate by ring-opening polymerization of β-propiolactone. In this case, a wide variety of potentially suitable substances is likewise listed, and this also comprises various nitrogen-comprising organic compounds, for example the potentially carcinogenic N,N-dimethylaniline, but these likewise do not show any unifying structural feature essential for advantageous usability as such a splitting catalyst.

Mentioned by way of example in U.S. Pat. No. 2,361,036 are merely thermolyses of poly-3-hydroxypropionate in which sodium carbonate is used as the splitting catalyst, which is associated with the disadvantages already described.

It was therefore an object of the present invention to provide a process, improved over the prior art processes, for preparation of acrylic acid by a thermolysis of poly-3-hydroxypropionate catalyzed by at least one active compound.

Accordingly, a process is provided for preparing acrylic acid by a thermolysis of poly-3-hydroxypropionate catalyzed by at least one molecular (i.e. non-salt, nonionic) organic active compound having at least one tertiary nitrogen atom which has a covalent bond to three different carbon atoms (to not more and not less than these three and not to any other atom type either), wherein the at least one molecular organic active compound
does not have any heteroatom other than carbon and hydrogen over and above nitrogen and oxygen,
does not have any nitrogen atom to which one or more than one hydrogen atom is covalently bonded,
has at most one oxygen atom to which a hydrogen atom is covalently bonded,
does not comprise any oxygen atom which has a covalent double bond to any of the three different carbon atoms (to which the at least one (respective) tertiary nitrogen atom has a covalent bond),
has neither a radical of an aromatic hydrocarbon nor a radical of a substituted aromatic hydrocarbon,
has a boiling point which, at a pressure of 1.0133·10⁵ Pa, is at least 150° C. and not more than 350° C., and
has a melting point which, at a pressure of 1.0133·10⁵ Pa, is ≦70° C.

Processes for preparing poly-3-hydroxypropionate as usable for the process according to the invention (suitable for the process according to the invention) are known in the prior art (more particularly in all the prior art detailed hereinafter in the present document).

For example, poly-3-hydroxypropionate (suitable for (all) the process(es) according to the invention) can be obtained by dehydrating polycondensation of 3-hydroxypropionic acid (cf., for example, Chinese Journal of Synthetic Chemistry, Vol. 15 (2007), No. 4, pages 452-453). Typical relative weight-average molecular weights M_{W} (i.e. based on the weight of atomic hydrogen) of poly-3-hydroxypropionate obtainable in this way may, for example, be 1000 to 20 000 (but also less or more).

The corresponding polydispersity Q (the ratio of weight-average relative molecular weight M_{W} to number-average relative molecular weight Mₙ (Q=M_{W}/Mₙ)) is generally at values of ≦2.5, frequently at values of ≦2. It is also possible to obtain polydispersities Q of ≦1.5.

U.S. Pat. No. 2,568,636, U.S. Pat. No. 2,361,036 and U.S. Pat. No. 3,002,017 A disclose preparing the polyester of 3-hydroxypropionic acid (one which is suitable for the process according to the invention) proceeding from β-propiolactone by ring-opening polymerization. Corresponding ring-opening polymerizations are also disclosed by WO 2011/163309 A2 and EP 688806 B1. According to the latter, the relative weight-average molecular weight M_{W} of poly-3-hydroxypropionate (suitable for all the processes according to the invention) obtainable in this way may, for example, be 5000 to 2 000 000, or 20 000 to 500 000, or 30 000 to 400 000. Relative weight-average molecular weights M_{w} above 100 000 are considered typical for the use of poly-3-hydroxypropionate contemplated in EP 688806 B1. The corresponding polydispersities Q are generally likewise at values of ≦2.5.

The thesis “Multi-Site Catalysis—Novel Strategies to Biodegradable Polyesters from Epoxides/CO and Macrocyclic Complexes as Enzyme Models” by Markus Allmendinger, University of Ulm (2003), discloses that, by carbonylating reaction of ethylene oxide dissolved in an aprotic solvent with carbon monoxide at elevated pressure, elevated temperature and in the presence of a catalyst system comprising at least one cobalt source, a product mixture comprising poly-3-hydroxypropionate is obtainable directly (i.e. without forming propiolactone (oxetan-2-one) as an intramolecular cyclic ester of β-hydroxypropionic acid (=3-hydroxypropionic acid) as an intermediate), from which the poly-3-hydroxypropionate can be removed by precipitation (for example by lowering the temperature and/or adding a precipitation liquid) and then employing one or more mechanical separating operations, for example filtration and/or centrifugation.

J. Am. Chem. Soc. 2002, 124, pages 5646-5647, DE 10137046 A1, WO 03/011941 A2 and J. Org. Chem. 2001, 66, pages 5424-5426 confirm these facts.

Typical relative weight-average molecular weights M_{W} of poly-3-hydroxypropionates (suitable for all the processes according to the invention) obtainable in the course of the aforementioned carbonylations of ethylene oxide may, for example, be 1000 to 20 000 or to 15 000, in many cases 2000 to 12 000, and frequently 3000 to 10 000 or 4000 to 10 000. In principle, however, higher and lower relative weight-average molecular weights M_{W} are also obtainable by this procedure. The corresponding polydispersity Q is generally at values of ≦2.5, frequently at values of 2. In many cases, Q is 1.5 to 1.8. However, it is also possible to establish polydispersities Q below 1.5 or below 1.4 (cf. DE 10137046 A1).

In the prior art preparation processes described to date, essentially a poly-3-hydroxypropionate homopolymer (homopolyester) is obtained.

In other words, an individual macromolecule of the respective poly-3-hydroxypropionate consists essentially exclusively of a structural section of the general formula (I) and forms a polyester of the general structure II

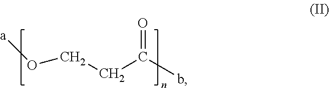

where n≧6 and a, b are a head group (a) bordering the polyester and an end group (b) bordering the polyester.

The nature of the respective head group/end group depends on the preparation process used in each case and on the preparation conditions employed in each case.

For example,

a may be

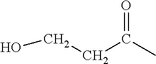

and
b may be

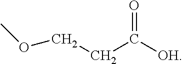

Alternatively,

a may be

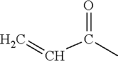

and
b may be

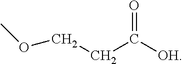

Normally, the relative molecular weight of a head group/end group is ≦150, usually ≦120 and generally ≦100.

According to the details so far, n in polyesters of the general structure II (and hence also in structural sections of the general formula I relevant in accordance with the invention) may, for example, be ≧6 and ≦30 000, or ≧8 and ≦25 000, or ≧10 and ≦20 000, or ≧15 and ≦15 000, or ≧20 and ≦10 000, or ≧25 and ≦8000, or ≧30 and ≦5000, or ≧40 and ≦2500, or ≧50 and ≦1500, or ≧60 and ≦1000, or ≧60 and ≦750, or ≧60 and ≦500, or ≧60 and ≦300, or ≧60 and ≦175, or ≧60 and ≦150, or ≧60 and ≦125, or ≧60 and ≦100.

In principle, however, a poly-3-hydroxypropionate copolymer is also useful for the process according to the invention (for all the processes according to the invention) (copolyester). Such a copolymer comprises, as well as structural sections of the general formula (I), also different structural sections. For example, such poly-3-hydroxypropionate copolymers are possible by the process for ring-opening polymerization of cyclic esters and/or cyclic ethers described in EP 688806 B1 when the molar proportion of β-propiolactone in the mixture of cyclic esters and cyclic ethers to be polymerized is only ≧80 mol %, or only 85 mol %, or only ≧90 mol %, or only ≧95 mol %, or only ≧98 mol %, or only ≧99 mol %. Useful cyclic esters other than 3-propiolactone include, for example, β-butyrolactone, pivalactone, δ-valerolactone and ε-caprolactone. Useful cyclic ethers other than β-propiolactone include, for example, ethylene oxide, propylene oxide and butylene oxide.

According to the teaching of WO 2011/100608 A1, poly-3-hydroxypropionate (suitable for all the processes according to the invention), however, can also be prepared either as a homopolymer or as a copolymer by a biotechnological route in genetically modified biological organisms (for example from sugars or from alternative “renewable” carbon sources to these). Useful biological organisms of this kind include, for example, bacteria, algae, yeasts, fungi or plants.

The relative weight-average molecular weight of biotechnologically produced poly-3-hydroxypropionate may be up to 100 000, or up to 200 000 or more.

The aforementioned relative weight-average molecular weight is normally ≧1000 or ≧5000.

The proportion by weight of structural sections of the general formula (I) in such “biotechnologically” obtainable poly-3-hydroxypropionate may, for example, be ≧40% by weight, or ≧50% by weight, or ≧60% by weight, or ≧70% by weight, or ≧80% by weight, or ≧90% by weight, or ≧95% by weight, or ≧97% by weight, or ≧98% by weight, or ≧99% by weight.

For the purpose of the inventive catalyzed thermolysis thereof, a biotechnologically produced poly-3-hydroxypropionate may either remain in the biological organism that produces it (in the total amount of the biological organisms that produce it=total amount of bioorganisms=in the “biomass”) or be extracted therefrom beforehand (cf. WO 2011/100608 A1).

If the poly-3-hydroxypropionate remains in the biomass during the inventive catalyzed thermolysis thereof, it is appropriate in application terms to substantially dry the biomass prior to the onset of thermolysis of the poly-3-hydroxypropionate (advantageously in application terms, a process for vacuum drying and/or for freeze-drying is employed in this regard). In principle, such drying of the biomass, however, may also be effected only in the course of the temperature increase required for the thermolysis (before the temperature at which the splitting sets in has been attained; this applies in a completely corresponding manner and generally for any poly-3-hydroxypropionate which is obtained in moist form in the course of preparation thereof and is to be split in accordance with the invention).

If the biomass comprises, for example, bacteria, it may be necessary to deactivate (to pasteurize or to sterilize) them prior to the relevant thermolysis (with respect to its biological characteristics). This can be accomplished, for example, by heating under pressure and with the optional use of steam, i.e. by “autoclaving” or “sterilizing”. It will be appreciated that the deactivation can also be accomplished with dry heat (“hot air sterilization”). Alternatively, the deactivation can also be undertaken by irradiating or by chemical methods.

If a catalyzed thermolysis (catalyzed in accordance with the invention) of biotechnologically produced poly-3-hydroxypropionate is undertaken still in the presence of the biomass, it is advantageous, prior to the thermolysis, to destroy the cell walls of those cells (for example the cell walls of the bacteria) in which it has been synthesized and/or in which it has been stored. Such destruction can be effected, for example, mechanically by the action of appropriate forces. For example, the biomass can be homogenized in a mixer with rotating blades (for example an Ultraturrax). Alternatively, the biological organisms (especially in the case of microorganisms) can also be triturated in a simple manner (for example in a mortar with sand or Al₂O₃, or with a pestle, or in a glass bead mill). In the case of action of sound waves (for example ultrasound), the cells are destroyed by constant collision (cavitation forces). A particularly preferred mechanical method for destruction of cell walls is the nitrogen decompression method. This involves enriching nitrogen in the cells at elevated gas pressures in accordance with Henry's law. A subsequent instantaneous pressure release can subsequently bring about the bursting of the cell walls.

Nonmechanical destruction processes are preferably employed in the case of cell walls which cannot be broken mechanically in a simple manner (for example in the case of yeast cells). Repeated freezing and thawing destroys the cell walls as a result of shear forces. Chemical (for example with toluene) and/or enzymatic lysis can destroy the cell membrane or cell wall. In addition, treatment with hypotonic buffer solutions can bring about the lysis of cells.

As a basic requirement, an active substance for use as a splitting catalyst in accordance with the invention should have maximum mass-specific catalytic action. In other words, a minimum use amount of the active substance should be sufficient to display the desired catalytic action.

In-house studies by the applicant have shown that this is the case for amines as molecular organic active compounds when the compound is a tertiary amine in the inventive sense. This means a molecular organic active compound which has at least one tertiary nitrogen atom which has a covalent bond to three different carbon atoms of the molecular organic active compound, without any oxygen atom bonded to any of these carbon atoms via a covalent double bond.

One reason for this is probably that primary and secondary amines can react with ester groups, as present in poly-3-hydroxypropionate to be split in accordance with the invention, to give amides. However, the nitrogen atom present in the amide group thereof is covalently bonded to a carbon atom which has a covalent double bond to an oxygen atom. The electron-attracting effect thereof, however, is prohibitive for usability as an efficient molecular organic active compound in the context of the present application.

For the purposes of maximum mass-specific catalytic splitting action, a molecular organic active compound to be used as a splitting catalyst in accordance with the invention, advantageously in accordance with the invention, has more than one tertiary nitrogen atom having a covalent bond to each of three different carbon atoms of the molecular organic active compound, with the proviso that none of these carbon atoms simultaneously has a covalent double bond to an oxygen atom. Favorably in accordance with the invention, any molecular organic active compound to be used as a splitting catalyst comprises at least two or at least three tertiary nitrogen atoms of this kind.

Most preferably, the relevant molecular organic active compound comprises only nitrogen atoms which are tertiary nitrogen atoms of the type detailed above.

The restriction to hydrogen, carbon, nitrogen and oxygen as possible atomic constituents of a molecular organic active compound suitable as a splitting catalyst in accordance with the invention ensures that this can optionally be fully combusted with residues remaining in the context of the relevant thermolysis, without any risk of formation of particularly problematic combustion gases.

Furthermore, the above restriction also limits, in a natural manner, side reactions which are unwanted in the context of the relevant thermolysis, and simultaneously promotes economically advantageous availability of the molecular organic active compound.

Advantageously in accordance with the invention, an inventive molecular organic active compound suitable as a splitting catalyst does not have any oxygen atom to which a hydrogen atom is covalently bonded. In this way, possible unwanted esterification of acrylic acid formed in the course of the thermolysis is counteracted.

The exclusion of radicals of an aromatic or of a substituted aromatic hydrocarbon ensures that molecular organic active compounds to be used as splitting catalysts in accordance with the invention are toxicologically comparatively safe compared to active compounds such as N,N-dimethylaniline, for example. This is particularly with a view to further use of the acrylic acid obtained in the course of an inventive thermolysis for preparation of polymers which find use in the hygiene sector.

The term “aromatic hydrocarbon” shall comprise both monocyclic aromatic hydrocarbons (e.g. benzene) and polycyclic aromatic hydrocarbons (which have at least two aromatic ring systems bonded to one another (e.g. naphthalene or biphenyl)). Substituted aromatic hydrocarbons derive from aromatic hydrocarbons by replacement of at least one hydrogen atom by a substituent (=an atom other than hydrogen or an (atom) group (=group of atoms chemically bonded to one another) other than a hydrogen atom) (examples of such substituted aromatic hydrocarbons are, for example, phenyl chloride (a hydrogen atom in the benzene is replaced by a chlorine atom) or toluene (a hydrogen atom in the benzene is replaced by the methyl group)).

The term “radical” expresses the fact that, in contrast to the aromatic hydrocarbon or to the substituted aromatic hydrocarbon, there is an unoccupied (free) covalent single bond therein, which may be localized either on an aromatic ring or on a substituent (e.g. —C₆H₅=phenyl radical or —CH₂—C₆H₅=benzyl radical, or C₆H₅—(C═O)—=benzoyl radical).

More preferably in accordance with the invention, the at least one molecular organic active compound does not have any aromatic ring system at all, i.e. no heteroaromatic ring either (the latter comprising at least one atom other than carbon in the aromatic ring).

The lower boiling point limit of the molecular organic active compounds suitable as a splitting catalyst in accordance with the invention (this boiling point at standard pressure is ≧150° C., better ≧160° C. or ≧170° C., advantageously ≧180° C., preferably ≧185° C., more preferably ≧190° C. and most preferably ≧195° C.) ensures that the inventive molecular organic active compounds, in the course of the thermolysis of poly-3-hydroxypropionate that they catalyze, need not normally necessarily be discharged from the splitting mixture with the acrylic acid formed in the splitting, but may generally remain in the splitting mixture (the latter can be promoted by a rectification column which is positioned atop a splitting reactor and is operated in reflux). By gradually adding fresh poly-3-hydroxypropionate to be split to the splitting mixture, it is possible in this case to make multiple (repeated) use of the action of one and the same splitting catalyst addition.

The upper boiling point limit of the molecular organic active compound suitable as a splitting catalyst in accordance with the invention (this boiling point at standard pressure is ≦350° C., preferably ≦345° C., better ≦340° C., advantageously ≦335° C., particularly advantageously ≦330° C. or ≦325° C., very particularly advantageously ≦320° C. or ≦315° C., even better ≦310° C. and at best ≦300° C., or ≦290° C., or ≦280° C., or ≦270° C., or ≦260° C., or ≦250° C. and ≦240° C., or ≦230° C., or ≦220° C.) opens up the possibility, after the catalyzed thermal splitting (catalyzed thermolysis) has ended, of removing the at least one molecular organic active compound used as a splitting catalyst in accordance with the invention subsequently from residues which generally remain in the relevant thermolysis (for example from remaining biomass), for example by distillative and/or rectificative means, optionally under reduced pressure, and thus obtaining it as a product of value for a process according to the invention in reutilizable form.

The melting point of molecular organic active compounds to be used as splitting catalysts in accordance with the invention, which is at comparatively low temperatures compared to the boiling point and is required in accordance with the invention (this melting point at standard pressure is ≦70° C., advantageously ≦60° C., particularly advantageously ≦50° C., better ≦40° C., preferably ≦30° C., more preferably ≦20° C. or ≦10° C., even more preferably ≦0° C. or ≦−10° C., and at best ≦−15° C.) is advantageous in that it ensures that the molecular active compound to be used in accordance with the invention as a splitting catalyst normally melts at a lower temperature than the poly-3-hydroxypropionate to be split itself and as a result can optionally function as a solvent or as a dispersant with respect to the poly-3-hydroxypropionate to be split. In the extreme case, the thermal splitting catalyzed in accordance with the invention (the thermolysis catalyzed in accordance with the invention) of the poly-3-hydroxypropionate can thus be effected from the solution thereof, or from the suspension thereof, or from the emulsion thereof, in the splitting catalyst. By employing a mixture of various molecular organic active compounds suitable as splitting catalysts in accordance with the invention, it is possible to bring about a melting point depression which is advantageous in the present context.

Moreover, a comparatively low melting point of a molecular organic active compound suitable as a splitting catalyst in accordance with the invention generally causes a comparatively low dynamic viscosity of the melt thereof, not only under the conditions of the thermolysis but also under customary conditions prior to the thermolysis. The latter is significant especially when the poly-3-hydroxypropionate to be split thermolytically itself has a comparatively high melting point (for example >200° C., or >250° C.). In these cases, the thermolysis of the poly-3-hydroxypropionate catalyzed in accordance with the invention can also be effected from the solid substance thereof. If the poly-3-hydroxypropionate in such a case can be sprayed homogeneously, for example, with a comparatively volatile splitting catalyst prior to the thermolysis, this is normally beneficial for a subsequently comparatively homogeneous profile of the thermolysis. Alternatively, solid poly-3-hydroxypropionate, for splitting purposes, can be impregnated in a comparatively simple manner with a volatile splitting catalyst, or be suspended therein.

In addition, a volatile splitting catalyst can also be applied in a simple manner to solid poly-3-hydroxypropionate to be split by stripping the splitting catalyst out of the liquid substance thereof with a carrier gas, and subsequently passing the carrier gas laden with the splitting catalyst through the solid poly-3-hydroxypropionate to be split in order to strip off the splitting catalyst on the surface therefrom again.

The above relationships are fully correspondingly also advantageous especially when the thermolysis of the poly-3-hydroxypropionate is undertaken, for example, from solid biomass.

Good wetting of solid poly-3-hydroxypropionate and a comparatively high flashpoint are further advantages normally possessed by molecular organic active compounds to be used as splitting catalysts in accordance with the invention.

It is generally a characteristic feature of molecular organic active compounds which are suitable as splitting catalysts in accordance with the invention and can advantageously combine the profile of properties detailed that the molar mass M thereof is ≧100 g/mol and ≦300 g/mol, advantageously ≧120 g/mol and ≦280 g/mol, preferably ≧140 g/mol and ≦260 g/mol, and more preferably ≧150 g/mol and ≦250 g/mol.

Inventive molecular organic active compounds particularly suitable as splitting catalysts for the process according to the invention (for all the thermolysis processes detailed in the present document, and on all poly-3-hydroxypropionates which are thermolytically splittable to give acrylic acid and are detailed in the present document) are, as an illustrative list, pentamethyldiethylenetriamine (M=173.30 g/mol; b.p.=199° C.; m.p.<−20° C.; purchasable as Lupragen® N301 from BASF SE), N,N,N′N-tetramethyl-1,6-hexanediamine (M=172.31 g/mol; b.p.=212° C., m.p.=−46° C.; purchasable as Lupragen® N500 from BASF SE), bis(2-dimethylaminoethyl)ether (M=160.3 g/mol; b.p.=189° C., m.p.=60° C.; purchasable as Lupragen® N205 from BASF SE), 2,2′-dimorpholinodiethyl ether (M=244.33 g/mol, b.p.=309° C.; m.p.=−28° C.; purchasable as Lupragen® N106 from BASF SE), N,N′-diethylethanolamine (M=117.19 g/mol; b.p.=161° C.; m.p.=−70° C.), N,N-dimethylcyclohexylamine (M=127.23 g/mol; b.p.=159° C.; m.p.=−60° C.; purchasable as Lupragen® N100 from BASF SE), N-methylimidazole (M=82.12 g/mol; b.p.=198° C.; m.p.=−2° C.; purchasable as Lupragen® NMI from BASF SE) and 1,2-dimethylimidazole (M=96.13 g/mol; b.p.=204° C.; m.p.=38° C.).

Among the molecular organic active compounds listed above by way of example as splitting catalysts particularly suitable in accordance with the invention, pentamethyldiethylenetriamine is once again preferred (especially for all the thermolysis processes detailed in the present document, and on all poly-3-hydroxypropionates which are thermolytically splittable to give acrylic acid and are detailed in the present document), since it combines the properties of a splitting catalyst favorable in accordance with the invention in a particularly favorable manner.

Based on the weight of the mass of the (a) poly-3-hydroxypropionate to be split to give acrylic acid in accordance with the invention, the weight of the mass of the at least one inventive catalytically active molecular active compound in the process according to the invention is generally 0.01 to 15% by weight, or 0.05 to 10% by weight, often 0.1 to 5% by weight, preferably 0.5 to 4% by weight, or 1.5 to 3.5% by weight.

Naturally, the use amount of splitting catalyst (of the at least one catalytically active molecular organic active compound) in the process according to the invention may also be above the values mentioned above. This is especially the case when the splitting catalyst simultaneously also functions as a solvent or as a dispersant for the poly-3-hydroxypropionate to be split. Particularly in these cases, the use amounts of splitting catalyst on a basis corresponding to that above may easily be up to 50% by weight, or up to 100% by weight, or up to 150% by weight, or up to 200% by weight, or up to 250% by weight, or up to 300% by weight, or up to 500% by weight or more.

The above is also easily the case when the process according to the invention for thermolysis of poly-3-hydroxypropionate is performed on poly-3-hydroxypropionate still present in biomass, which for this purpose, advantageously for application purposes, may be slurried in the at least one molecular organic active compound for use as a splitting catalyst in accordance with the invention.

According to the melting point and solubility of the poly-3-hydroxypropionate, the process according to the invention for catalyzed thermal splitting thereof (for catalyzed thermolysis thereof) can be effected with formation of acrylic acid from the solid substance thereof, or from the melt thereof, or from the solution thereof in a solvent (for example an organic liquid), or from the suspension thereof in a (for example organic) liquid (in a dispersant) or from the emulsion thereof in a (for example organic) liquid (in a dispersant), or from the biomass thereof which comprises the poly-3-hydroxypropionate and may optionally be slurried in a (for example organic) liquid (in a slurrying agent).

The boiling point (based on standard pressure) of such a solvent, dispersant or slurrying agent is, advantageously in application terms, well (for example at least 20° C., better at least 40° C., even better at least 50° C. or at least 60° C., preferably at least 80° C. and more preferably at least 100° C.) above the boiling temperature of acrylic acid on a corresponding basis (=141° C.).

Useful such (for example organic) solvents, or dispersants, or slurrying agents, include, for example, ionic liquids, oligomeric (particularly di- to hexameric) Michael adducts (addition products) of acrylic acid onto itself and onto the addition products which form (as typically arise in the course of conventional preparation of acrylic acid (especially, for example, as bottom products in the case of retifications of acrylic acids or as residues in the case of storage of acrylic acid)), or molecular organic liquids such as dimethyl sulfoxide, N-methyl-2-pyrrolidone, dialkylformamide, relatively long-chain paraffinic hydrocarbons, relatively long-chain alkanols, γ-butyrolactone, ethylene carbonate, diphenyl ether, diglyme (=diethylene glycol dimethyl ether), triglyme (=triethylene glycol dimethyl ether), tetraglyme (=tetraethylene glycol dimethyl ether), biphenyl, tricresyl phosphate, dimethyl phthalate and/or diethyl phthalate, among which preference is given in accordance with the invention to the nonaromatic liquids.

The proportion by weight of the poly-3-hydroxypropionate in such a splitting mixture also comprising solvent, or dispersant, or slurrying agent, may, based on the weight of the total mass of the splitting mixture, be less than 95% by weight, or less than 90% by weight, or less than 80% by weight, or less than 70% by weight, or less than 60% by weight, or less than 50% by weight, or less than 40% by weight, or less than 30% by weight, or less than 20% by weight, or less than 10% by weight. In general, this proportion by weight is, however, ≧5% by weight.

The proportion by weight of the poly-3-hydroxypropionate in dry biomass may have corresponding values. In favorable cases, however, it is at values of ≧95% by weight (cf., for example, WO 2011/100608).

Irrespective of whether the poly-3-hydroxypropionate is present in the splitting mixture in the form of the melt thereof, or dissolved in a solvent, or dispersed in a dispersant as a suspension or as an emulsion (i.e. suspended or emulsified), or in slurried form as a constituent of biomass in a slurrying agent, the at least one molecular organic active compound added as a splitting catalyst is preferably present dissolved in the splitting mixture (in the melt, in the solvent, in the dispersant, or in the slurrying agent).

In general, presence of solvent, or dispersant, or slurrying agent, however, reduces the splitting rate under otherwise identical conditions.

The position of the melting point (based on standard pressure) of poly-3-hydroxypropionate depends especially on the relative weight-average molecular weight and the polydispersity Q thereof.

In the case of values of the weight-average relative molecular weight M_{W} of poly-3-hydroxypropionate in the range from 1000 to 20 000, the corresponding melting point based on standard pressure (at customary polydispersities) is normally at values of ≦150° C., usually ≦100° C.

Even in the case of values for M_{W} of up to 100 000, or up to 150 000, the melting point of the poly-3-hydroxypropionate based on standard pressure (at customary polydispersities) is still at values of 200° C.

In these aforementioned cases, the thermolysis process according to the invention is therefore generally advantageously executed from the melt of the poly-3-hydroxypropionate. It is advantageous in this case that the at least one molecular organic active compound or melt thereof to be added (additionally used) as a splitting catalyst in accordance with the invention dissolves completely in the melt in the catalytically active amount thereof to be added which is required in each case, or mixes completely and homogeneously with the melt of the poly-3-hydroxypropionate to be thermally split.

Otherwise, the process according to the invention for catalyzed thermolysis of poly-3-hydroxypropionate can be performed (executed) as described in the known prior art splitting processes (for example the prior art acknowledged in the present document).

In other words, splitting temperatures typically to be employed (temperatures at which the thermolysis is performed, possessed by the poly-3-hydroxypropionate or melt, solution, suspension, emulsion thereof, the biomass comprising it or the slurries of the biomass comprising it in the course of thermolysis) may vary within the range from 50 to 400° C., or within the range from 75° C. to 350° C., or within the range from 100 to 300° C. Advantageously in accordance with the invention, the splitting temperatures employed (the thermolysis temperatures, the temperatures at which the thermolysis is performed) will be 150 to 220° C. and more preferably 160 to 200° C.

Equally, the working pressure during the inventive thermolysis of the poly-3-hydroxypropionate (in the gas atmosphere) may be either at standard pressure (=1.0133·10⁵ Pa) or above or below standard pressure. In other words, the working pressure may, for example, be 10² to 10⁷ Pa, or 10³ to 10⁶ Pa, or 2·10³ to 5·10⁵ Pa, or 5·10³ to 3·10⁵ Pa.

If the working pressure is below standard pressure (for example at pressures down to 10² Pa or less), the acrylic acid formed in the splitting follows the pressure gradient present and is withdrawn continuously from the liquid splitting mixture in this manner.

If the working pressure is at or above standard pressure (for example at pressures up to 10⁷ Pa or more), the acrylic acid formed in the splitting, appropriately in application terms, can be continuously stripped out of the splitting mixture, for example in liquid form (which may, for example, also be the exclusive melt of poly-3-hydroxypropionate (P3HP)), with the aid of a stripping gas (for example molecular nitrogen, noble gas, carbon dioxide, air, lean air (preferred; molecular oxygen-depleted air (generally <6% by vol. of O₂))).

The measure of stripping can also advantageously be partly employed in the context of splitting under reduced pressure.

It will be appreciated that the acrylic acid formed in the course of splitting can also be distilled out of the splitting mixture, for example in liquid form, in a conventional manner following the corresponding temperature gradient.

If, for example, the gas stream which comprises the acrylic acid formed in the splitting and is flowing away from the splitting mixture, for example in liquid form, is conducted in countercurrent to descending reflux liquid through a rectification column on top of a splitting reactor, the acrylic acid can be removed in elevated purity from the liquid splitting mixture (this is advantageous, for example, when the poly-3-hydroxypropionate to be split thermolytically in accordance with the invention is not a homopolymer but a copolymer). Additional subsequent employment of any thermal separation processes can result in purification of the acrylic acid to any desired purity.

All such splitting operations on poly-3-hydroxypropionate by the action of elevated temperatures are summarized in this document by the term “thermolysis” or “pyrolysis” of poly-3-hydroxypropionate.

The process according to the invention for catalyzed thermolysis of poly-3-hydroxypropionate is applicable, inter alia, to all poly-3-hydroxypropionates detailed in this document, even if they do not have a vinylic head group and/or a vinylic end group (a vinylic head group and end group shall be understood to mean, respectively, a head group and end group which have at least one ethylenically unsaturated double bond between two carbon atoms).

It should also be emphasized that poly-3-hydroxypropionate which is prepared by carbonylation of ethylene oxide dissolved in an aprotic solvent with CO in the presence of a cobalt-comprising catalyst system at elevated pressure and elevated temperature, as described in the processes of the prior art acknowledged in this document, is subjected prior to the inventive catalyzed thermolysis thereof to a decobaltization by, for example, washing with an aqueous solution, preferably with a Brønsted-acidic aqueous solution (the reference basis for the property “Brønsted acid” in this document is 25° C. and standard pressure, and water as the co-reactant for the Brønsted acid; in other words, the addition of a Brønsted acid to water (at 25° C. and standard pressure) gives an aqueous solution which, under the conditions mentioned, has a lower pH than pure water; such aqueous solutions are what is meant by the expression “Brønsted-acidic aqueous solution”), and/or by precipitation from the product mixture comprising it with an aqueous solution, preferably with a Brønsted-acidic aqueous solution. Advantageously, the washing and/or precipitation is effective in the presence of one or more oxidizing agents for Co in oxidation states <+2. Appropriately in application terms, the precipitation and/or washing is therefore effected, for example, under air. The reason for this measure is that the applicant has found that presence of cobalt impairs the inventive catalyzed thermolysis.

Additional use of at least one inventive organic molecular active compound as a splitting catalyst in the inventive thermolysis not only enables performance of the thermolysis at relatively low temperatures, but also ensures, under given thermolysis conditions, normally especially also an elevated space-time yield of acrylic acid (the at least one molecular organic active compound, under given conditions, generally improves both the splitting rate and the selectivity of target product formation (of acrylic acid formation)).

In order to optionally counteract any unwanted free-radical polymerization of acrylic acid formed in the inventive thermolysis, appropriate polymerization inhibitors can additionally be added to the poly-3-hydroxypropionate to be split thermolytically, or to the melt thereof, or to the solution thereof in a solvent, or to the emulsion thereof in a dispersant, or to the suspension thereof in a dispersant, or to the biomass comprising the poly-3-hydroxypropionate, or to the slurry of the biomass comprising the poly-3-hydroxypropionate in a slurrying agent.

Useful polymerization inhibitors of this kind in principle include all of those which are recommended in the prior art for the purpose of inhibiting free-radical polymerization of acrylic acid in the liquid phase. Useful polymerization inhibitors of this kind include alkylphenols, such as ortho-, meta- or para-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol and 2-methyl-4-tert-butylphenol, hydroxyphenols such as hydroquinone, catechol, resorcinol, 2-methylhydroquinone and 2,5-di-tert-butylhydroquinone, aminophenols, for example para-aminophenol, nitrosophenols, for example para-nitrosophenol, alkoxyphenols such as 2-methoxyphenol, 2-ethoxyphenol, 4-methoxyphenol (hydroquinone monomethyl ether) and mono- or di-tert-butyl-4-methoxyphenol, tocopherols, for example α-tocopherol, N-oxyls such as 4-hydroxy-2,2,6,6-tetramethylpiperidine N-oxyl, 2,2,6,6-tetramethylpiperidine N-oxyl, 4,4′,4″-tris(2,2,6,6-tetramethylpiperidine N-oxyl) phosphite or 3-oxo-2,2,5,5-tetramethyl-pyrrolidine N-oxyl, aromatic amines or phenylenediamines, for example N,N-diphenylamine, N-nitrosodiphenylamine and N,N′-dialkyl-para-phenylenediamine, where the alkyl radicals may be the same or different and each independently consist of 1 to 4 carbon atoms and may be straight-chain or branched, hydroxylamines, for example N,N-diethylhydroxylamine, phosphorus compounds, for example triphenylphosphine, triphenyl phosphite, hypophosphorous acid or triethyl phosphite, sulfur compounds, for example diphenyl sulfide or phenothiazine, and all aforementioned inhibitors optionally in combination with metal salts, for example the chlorides, dithiocarbonates, sulfates, salicylates or acetates of copper, manganese, cerium, nickel and/or chromium.

It is also possible to use different mixtures of the polymerization inhibitors mentioned. The polymerization inhibitors used are preferably phenothiazine and/or hydroquinone monomethyl ether. In addition, the aforementioned polymerization inhibitors can be supported by a molecular oxygen-comprising gas (for example air or nitrogen-diluted air (advantageously lean air=air depleted of molecular oxygen, the molecular oxygen content of which is typically <6% by volume)). Appropriately in application terms, the explosion limits of gaseous mixtures comprising acrylic acid and oxygen are noted (cf., for example, WO 2004/007405 A1). For example, the above support can be effected by stripping the acrylic acid formed in the splitting continuously out of the splitting mixture with the aid of a molecular oxygen-comprising stripping gas (such a stripping operation can be effected at reduced pressure, standard pressure, or else at working pressures above standard pressure).

According to the polymerization inhibitor (or mixture of polymerization inhibitors) used, the use amount thereof, based on the content of poly-3-hydroxypropionate in the splitting mixture, will be 10 to 1000 ppm by weight, frequently 50 to 500 ppm by weight and in many cases 150 to 350 ppm by weight.

Apart from the above-described possible additional use of a molecular oxygen-comprising stripping gas and optional promotion of polymerization inhibitors by a molecular oxygen-comprising gas, the inventive catalyzed thermolysis, appropriately in application terms, is executed with substantial exclusion of molecular oxygen, in order to prevent unwanted oxidation (especially unwanted full combustion) of organic components present in the thermolysis.

It should also be emphasized that the process according to the invention can be performed continuously or batchwise.

The acrylic acid can be converted from the acrylic acid-comprising gas phase obtained in the thermolysis of poly-3-hydroxypropionate catalyzed in accordance with the invention to the liquid phase in a manner known per se, by absorptive and/or condensative measures. In general, this liquid phase may already be the acrylic acid which is obtainable in accordance with the invention and is suitable for further uses (for example free-radical polymerizations) (especially when the acrylic acid thus obtained is not stored intermediately prior to the further use thereof in a free-radically initiated polymerization, the aforementioned conversion to the liquid phase will advantageously be undertaken without additional use of polymerization inhibitors which impair any (later) free-radically initiated polymerization).

With application of one or more thermal separation processes (such thermal separation processes may especially be rectification, extraction, desorption, distillation, stripping, absorption, azeotropic rectification and/or crystallization) to the acrylic acid-comprising liquid phase, the acrylic acid from the liquid phase can also be purified to any purity as required (for example analogously as described in documents DE 10243625 A1, DE 10332758 A1, DE 102007004960 A1 and DE 102012204436 A1, and the prior art cited in these documents).

A suitable preferred thermal separation process is the process of crystallization. Within the crystallizative separation processes, the process of suspension crystallization is preferentially employable for the aforementioned purpose (for example analogously as described in DE 102007043759 A1, DE 102008042008 A1 and DE 102008042010 A1, and the prior art cited in these documents).

The removal of the suspension crystals from the crystal suspension is, appropriately in application terms, undertaken in a wash melt wash column (cf. WO 01/77056 A1; the wash liquid used is the melt of acrylic acid crystals already purified correspondingly), preferably in a hydraulic wash melt wash column (analogously as described, for example, in WO 01/77056 A1, WO 02/09839 A1, WO 03/041832 A1, WO 2006/111565 A2, WO 2010/094637 A1 and WO 2011/045356 A1, and the prior art cited in these documents).

Incidentally, the inventive splitting of the poly-3-hydroxypropionate can be performed on the industrial scale either batchwise or continuously.

Appropriately in application terms, a continuous process regime may be configured as follows. The splitting reactor used is the bottom space of a separating column comprising separating internals (useful separating internals include, for example, mass transfer trays such as dual-flow trays; in principle, the separating column may also be empty, i.e. not have any separating internals). The liquid splitting mixture (which may be a melt, a solution, a suspension, a slurry or an emulsion) is supplied in the lower third of the separating column (in principle, the supply may also be effected directly into the bottom space; such a supply may in principle also be effected “in solid form”).

Below the feed point (advantageously from the bottom space), by means of a pump, a liquid stream (which may optionally also be a suspension or slurry) is withdrawn continuously and recycled by means of an indirect heat exchanger back into the separating column below the feed point of the splitting mixture. In the course of flow through the indirect heat exchanger, the thermal energy required for the thermolysis is supplied. Advantageously in application terms, the indirect heat exchanger is a forced circulation flash heat transferer.

At the top or via a side draw, the acrylic acid can be conducted out of the separating column. If the separating column has separating internals, condensate formation is brought about in the top region of the separating column and a portion of the condensate formed is conducted in the separating column descending as reflux liquid in countercurrent to the acrylic acid ascending in the separating column (for example conducted by a stripping gas and/or following the pressure gradient in the case of reduced top pressure). As an outlet for the highest-boiling secondary components, a portion of the bottoms liquid is discharged continuously and sent to the disposal (for example incineration) thereof.

If the inventive thermolysis is performed from the solid substance of the poly-3-hydroxypropionate or from the solid biomass comprising it (preferably dry biomass), it is appropriate in application terms to perform the process according to the invention in a heated rotary tube oven, through which a stripping gas advantageously flows, the latter discharging the acrylic acid formed. It is possible in this case to work either batchwise or continuously. In continuous operation, the material to be thermolyzed in accordance with the invention and the stripping gas are appropriately conducted through the rotary tube oven in countercurrent.

What is advantageous about acrylic acid which has been prepared by the inventive procedure (or originates from an inventive preparation) and has been converted, for example by absorptive and/or condensative measures, from the gas phase obtained in the thermolysis of the poly-3-hydroxypropionate to the condensed (preferably liquid) phase is that it does not have the fingerprint of low molecular weight aldehydes present therein as impurities which is typical of acrylic acid prepared by heterogeneously catalyzed partial oxidations of C₃ precursor compounds (e.g. propylene, propane, acrolein, glycerol, propionic acid, propanol, etc.) (cf., for example, DE 102011076931 A1).

These impurities are found to be extremely disruptive in the case of use of the acrylic acid and/or of the conjugate (Brønsted) base thereof for preparation of polymers by free-radically initiated polymerization, optionally in a mixture with other mono- or polyunsaturated (for example ethylenically) compounds, even in very small amounts (1 to 10 ppm by weight based on the weight of the mass of acrylic acid) (for example, they can undesirably retard the free-radically initiated polymerization or hinder or impair the preparation of polymer with particularly high molecular weight as a result of their “regulating action”).

Accordingly, particularly advantageous processes for inventive preparation of acrylic acid are those followed by a process for free-radical polymerization in which the acrylic acid prepared, as such and/or in the form of its conjugate base (what is meant here is the conjugate Brønsted base, the acrylate anion), optionally in a mixture with other mono- and/or polyunsaturated compounds, is polymerized to polymer with free-radical initiation.

This is especially true when the process for free-radical polymerization is a process for producing a water-“superabsorbent” polymer, as used, for example, in hygiene articles such as diapers (cf. DE 102011076931 A1 and the prior art cited in the same document).

Accordingly, the present invention comprises especially the following inventive embodiments:
1. A process for preparing acrylic acid by a thermolysis of poly-3-hydroxypropionate catalyzed by at least one molecular organic active compound having at least one tertiary nitrogen atom which has a covalent bond to three different carbon atoms in the molecular organic active compound, wherein the at least one molecular organic active compound
   does not have any heteroatom other than carbon and hydrogen over and above nitrogen and oxygen,
   does not have any nitrogen atom to which one or more than one hydrogen atom is covalently bonded,
   has at most one oxygen atom to which a hydrogen atom is covalently bonded,
   does not comprise any oxygen atom which has a covalent double bond to any of the three different carbon atoms,
   has neither a radical of an aromatic hydrocarbon nor a radical of a substituted aromatic hydrocarbon,
   has a boiling point which, at a pressure of 1.0133·10⁵ Pa, is at least 150° C. and not more than 350° C., and
   has a melting point which, at a pressure of 1.0133·10⁵ Pa, is ≦70° C.
2. The process according to embodiment 1, wherein the at least one molecular organic active compound comprises more than one tertiary nitrogen atom which has a covalent bond to each of three different carbon atoms of the molecular organic active compounds, with the proviso that none of these carbon atoms simultaneously has a covalent double bond to any oxygen atom.
3. The process according to embodiment 2, wherein the at least one molecular organic active compound comprises at least two tertiary nitrogen atoms which have a covalent bond to each of three different carbon atoms of the molecular organic active compounds, with the proviso that none of these carbon atoms simultaneously has a covalent double bond to any oxygen atom.
4. The process according to embodiment 2 or 3, wherein the at least one molecular organic active compound comprises at least three tertiary nitrogen atoms which have a covalent bond to each of three different carbon atoms of the molecular organic active compounds, with the proviso that none of these carbon atoms simultaneously has a covalent double bond to any oxygen atom.
5. The process according to any of embodiments 1 to 4, wherein the at least one molecular organic active compound comprises only tertiary nitrogen atoms which have a covalent bond to each of three different carbon atoms of the molecular organic active compounds, with the proviso that none of these carbon atoms simultaneously has a covalent double bond to any oxygen atom.
6. The process according to any of embodiments 1 to 5, wherein the at least one molecular organic active compound does not have any oxygen atom to which a hydrogen atom is covalently bonded.
7. The process according to any of embodiments 1 to 6, wherein the at least one molecular organic active compound has a boiling point which, at a pressure of 1.0133·10⁵ Pa, is at least 160° C.
8. The process according to any of embodiments 1 to 6, wherein the at least one molecular organic active compound has a boiling point which, at a pressure of 1.0133·10⁵ Pa, is at least 170° C.
9. The process according to any of embodiments 1 to 6, wherein the at least one molecular organic active compound has a boiling point which, at a pressure of 1.0133·10⁵ Pa, is at least 180° C.
10. The process according to any of embodiments 1 to 6, wherein the at least one molecular organic active compound has a boiling point which, at a pressure of 1.0133·10⁵ Pa, is at least 185° C.
11. The process according to any of embodiments 1 to 6, wherein the at least one molecular organic active compound has a boiling point which, at a pressure of 1.0133·10⁵ Pa, is at least 190° C.
12. The process according to any of embodiments 1 to 6, wherein the at least one molecular organic active compound has a boiling point which, at a pressure of 1.0133·10⁵ Pa, is at least 195° C.
13. The process according to any of embodiments 1 to 12, wherein the at least one molecular organic active compound has a boiling point which, at a pressure of 1.0133·10⁵ Pa, is not more than 345° C.
14. The process according to any of embodiments 1 to 13, wherein the at least one molecular organic active compound has a boiling point which, at a pressure of 1.0133·10⁵ Pa, is not more than 340° C.
15. The process according to any of embodiments 1 to 14, wherein the at least one molecular organic active compound has a boiling point which, at a pressure of 1.0133·10⁵ Pa, is not more than 335° C.
16. The process according to any of embodiments 1 to 15, wherein the at least one molecular organic active compound has a boiling point which, at a pressure of 1.0133·10⁵ Pa, is not more than 330° C.
17. The process according to any of embodiments 1 to 16, wherein the at least one molecular organic active compound has a boiling point which, at a pressure of 1.0133·10⁵ Pa, is not more than 320° C.
18. The process according to any of embodiments 1 to 17, wherein the at least one molecular organic active compound has a boiling point which, at a pressure of 1.0133·10⁵ Pa, is not more than 310° C.
19. The process according to any of embodiments 1 to 18, wherein the at least one molecular organic active compound has a boiling point which, at a pressure of 1.0133·10⁵ Pa, is not more than 300° C.
20. The process according to any of embodiments 1 to 19, wherein the at least one molecular organic active compound has a boiling point which, at a pressure of 1.0133·10⁵ Pa, is not more than 290° C.
21. The process according to any of embodiments 1 to 20, wherein the at least one molecular organic active compound has a boiling point which, at a pressure of 1.0133·10⁵ Pa, is not more than 270° C.
22. The process according to any of embodiments 1 to 21, wherein the at least one molecular organic active compound has a boiling point which, at a pressure of 1.0133·10⁵ Pa, is not more than 250° C.
23. The process according to any of embodiments 1 to 22, wherein the at least one molecular organic active compound has a boiling point which, at a pressure of 1.0133·10⁵ Pa, is not more than 240° C.
24. The process according to any of embodiments 1 to 23, wherein the at least one molecular organic active compound has a boiling point which, at a pressure of 1.0133·10⁵ Pa, is not more than 230° C.
25. The process according to any of embodiments 1 to 24, wherein the at least one molecular organic active compound has a boiling point which, at a pressure of 1.0133·10⁵ Pa, is not more than 220° C.
26. The process according to any of embodiments 1 to 25, wherein the at least one molecular organic active compound has a melting point which, at a pressure of 1.0133·10⁵ Pa, is ≦60° C.
27. The process according to any of embodiments 1 to 25, wherein the at least one molecular organic active compound has a melting point which, at a pressure of 1.0133·10⁵ Pa, is ≦50° C.
28. The process according to any of embodiments 1 to 25, wherein the at least one molecular organic active compound has a melting point which, at a pressure of 1.0133·10⁵ Pa, is ≦40° C.
29. The process according to any of embodiments 1 to 25, wherein the at least one molecular organic active compound has a melting point which, at a pressure of 1.0133·10⁵ Pa, is ≦30° C.
30. The process according to any of embodiments 1 to 25, wherein the at least one molecular organic active compound has a melting point which, at a pressure of 1.0133·10⁵ Pa, is ≦20° C.
31. The process according to any of embodiments 1 to 25, wherein the at least one molecular organic active compound has a melting point which, at a pressure of 1.0133·10⁵ Pa, is ≦10° C.
32. The process according to any of embodiments 1 to 25, wherein the at least one molecular organic active compound has a melting point which, at a pressure of 1.0133·10⁵ Pa, is ≦0° C.
33. The process according to any of embodiments 1 to 25, wherein the at least one molecular organic active compound has a melting point which, at a pressure of 1.0133·10⁵ Pa, is ≦−10° C.
34. The process according to any of embodiments 1 to 25, wherein the at least one molecular organic active compound has a melting point which, at a pressure of 1.0133·10⁵ Pa, is ≦−15° C.
35. The process according to any of embodiments 1 to 34, wherein the molar mass M of the at least one molecular organic active compound is 100 g/mol and ≦300 g/mol.
36. The process according to embodiment 35, wherein M≧120 g/mol and ≦280 g/mol.
37. The process according to embodiment 35 or 36, wherein M≧140 g/mol and ≦260 g/mol.
38. The process according to any of embodiments 35 to 37, wherein M≧150 g/mol and ≦250 g/mol.
39. The process according to embodiment 1, wherein the at least one molecular active compound is a molecular active compound from the group consisting of pentamethyldiethylenetriamine, N,N,N′,N′-tetramethyl-1,6-hexanediamine, bis(2-dimethylaminoethyl)ether, 2,2′-dimorpholinodiethyl ether, N,N′-diethylethanolamine, N,N-dimethylcyclohexylamine, N-methylimidazole and 1,2-dimethylimidazole.
40. The process according to any of embodiments 1 to 39, wherein the catalyzed thermolysis of the poly-3-hydroxypropionate, based on the weight of the mass thereof, is effected (catalyzed) by 0.01 to 15% by weight of the at least one molecular organic active compound.
41. The process according to any of embodiments 1 to 40, wherein the catalyzed thermolysis of the poly-3-hydroxypropionate, based on the weight of the mass thereof, is effected (catalyzed) by 0.05 to 10% by weight of the at least one molecular organic active compound.
42. The process according to any of embodiments 1 to 41, wherein the catalyzed thermolysis of the poly-3-hydroxypropionate, based on the weight of the mass thereof, is effected (catalyzed) by 0.1 to 5% by weight of the at least one molecular organic active compound.
43. The process according to any of embodiments 1 to 42, wherein the catalyzed thermolysis of the poly-3-hydroxypropionate, based on the weight of the mass thereof, is effected (catalyzed) by 0.5 to 4% by weight of the at least one molecular organic active compound.
44. The process according to any of embodiments 1 to 43, wherein the catalyzed thermolysis of the poly-3-hydroxypropionate, based on the weight of the mass thereof, is effected (catalyzed) by 1.5 to 3.5% by weight of the at least one molecular organic active compound.
45. The process according to any of embodiments 1 to 39, wherein the catalyzed thermolysis of the poly-3-hydroxypropionate, based on the weight of the mass thereof, is effected (catalyzed) by up to 50% by weight of the at least one molecular organic active compound.
46. The process according to any of embodiments 1 to 39, wherein the catalyzed thermolysis of the poly-3-hydroxypropionate, based on the weight of the mass thereof, is effected (catalyzed) by up to 100% by weight of the at least one molecular organic active compound.
47. The process according to any of embodiments 1 to 39, wherein the catalyzed thermolysis of the poly-3-hydroxypropionate, based on the weight of the mass thereof, is effected (catalyzed) by up to 150% by weight of the at least one molecular organic active compound.
48. The process according to any of embodiments 1 to 39, wherein the catalyzed thermolysis of the poly-3-hydroxypropionate, based on the weight of the mass thereof, is effected (catalyzed) by up to 200% by weight of the at least one molecular organic active compound.
49. The process according to any of embodiments 1 to 39, wherein the catalyzed thermolysis of the poly-3-hydroxypropionate, based on the weight of the mass thereof, is effected (catalyzed) by up to 300% by weight of the at least one molecular organic active compound.
50. The process according to any of embodiments 1 to 39, wherein the catalyzed thermolysis of the poly-3-hydroxypropionate, based on the weight of the mass thereof, is effected (catalyzed) by up to 500% by weight of the at least one molecular organic active compound.
51. The process according to any of embodiments 1 to 50, wherein the process for catalyzed thermolysis of the poly-3-hydroxypropionate is effected from the solid substance thereof, or from the melt thereof, or from the solution thereof in an organic liquid as a solvent, or from the suspension thereof in an organic liquid as a dispersant, or from the emulsion thereof in an organic liquid as a dispersant, or from a biomass comprising it, or from a slurry of the biomass comprising it in an organic solvent as a slurrying agent.
52. The process according to embodiment 51, wherein the boiling point of the organic liquid, based on a pressure of 1.0133·10⁵ Pa, is at least 20° C. above the boiling temperature of acrylic acid on a corresponding basis.
53. The process according to embodiment 51, wherein the boiling point of the organic liquid, based on a pressure of 1.0133·10⁵ Pa, is at least 40° C. above the boiling temperature of acrylic acid on a corresponding basis.
54. The process according to embodiment 51, wherein the boiling point of the organic liquid, based on a pressure of 1.0133·10⁵ Pa, is at least 60° C. above the boiling temperature of acrylic acid on a corresponding basis.
55. The process according to embodiment 51, wherein the boiling point of the organic liquid, based on a pressure of 1.0133·10⁵ Pa, is at least 80° C. above the boiling temperature of acrylic acid on a corresponding basis.
56. The process according to embodiment 51, wherein the boiling point of the organic liquid, based on a pressure of 1.0133·10⁵ Pa, is at least 100° C. above the boiling temperature of acrylic acid on a corresponding basis.
57. The process according to embodiment 51, wherein the organic liquid is selected from the group consisting of ionic liquids, oligomeric (particularly di- to hexameric) Michael adducts of acrylic acid onto itself and onto the addition products formed, dimethyl sulfoxide, N-methyl-2-pyrrolidone, dialkylformamide, relatively long-chain paraffinic hydrocarbons, relatively long-chain alkanols, γ-butyrolactone, ethylene carbonate, diphenyl ether, diglyme, triglyme, tetraglyme, biphenyl, tricresyl phosphate, dimethyl phthalate and/or diethyl phthalate.
58. The process according to any of embodiments 51 to 57, wherein the proportion by weight of the poly-3-hydroxypropionate in the solution, or in the suspension, or in the emulsion, or in the biomass, or in the slurry of the biomass, is at least 5 to at least 95% by weight.
59. The process according to any of embodiments 51 to 58, wherein the proportion by weight of the poly-3-hydroxypropionate in the solution, or in the suspension, or in the emulsion, or in the biomass, or in the slurry of the biomass, is at least 10 to at least 90% by weight.
60. The process according to any of embodiments 51 to 59, wherein the proportion by weight of the poly-3-hydroxypropionate in the solution, or in the suspension, or in the emulsion, or in the biomass, or in the slurry of the biomass, is at least 15 to at least 85% by weight.
61. The process according to any of embodiments 51 to 60, wherein the proportion by weight of the poly-3-hydroxypropionate in the solution, or in the suspension, or in the emulsion, or in the biomass, or in the slurry of the biomass, is at least 20 to at least 80% by weight.
62. The process according to any of embodiments 51 to 61, wherein the proportion by weight of the poly-3-hydroxypropionate in the solution, or in the suspension, or in the emulsion, or in the biomass, or in the slurry of the biomass, is at least 30 to at least 70% by weight.
63. The process according to any of embodiments 51 to 62, wherein the proportion by weight of the poly-3-hydroxypropionate in the solution, or in the suspension, or in the emulsion, or in the biomass, or in the slurry of the biomass, is at least 40 to at least 60% by weight.
64. The process according to any of embodiments 51 to 63, wherein the at least one organic active compound is present dissolved in the melt of the poly-3-hydroxypropionate or in the organic liquid.
65. The process according to any of embodiments 1 to 64, wherein the poly-3-hydroxypropionate in the course of thermolysis has a temperature of 50 to 400° C.
66. The process according to any of embodiments 1 to 65, wherein the poly-3-hydroxypropionate in the course of thermolysis has a temperature of 75 to 350° C.
67. The process according to any of embodiments 1 to 66, wherein the poly-3-hydroxypropionate in the course of thermolysis has a temperature of 100 to 300° C.
68. The process according to any of embodiments 1 to 67, wherein the poly-3-hydroxypropionate in the course of thermolysis has a temperature of 150 to 220° C.
69. The process according to any of embodiments 1 to 68, wherein the poly-3-hydroxypropionate in the course of thermolysis has a temperature of 160 to 200° C.
70. The process according to any of embodiments 1 to 69, which is performed at atmospheric pressure, above atmospheric pressure or below atmospheric pressure.
71. The process according to any of embodiments 1 to 70, which is performed at a working pressure of 10² to 10⁷ Pa.
72. The process according to any of embodiments 1 to 71, which is performed at a working pressure of 10³ to 10⁶ Pa.
73. The process according to any of embodiments 1 to 72, which is performed at a working pressure of 2·10³ to 5·10⁵ Pa.
74. The process according to any of embodiments 1 to 73, which is performed at a working pressure of 5·10³ to 3·10⁵ Pa.
75. The process according to any of embodiments 1 to 74, wherein the acrylic acid formed in the thermolysis is discharged continuously from the thermolysis with the aid of a stripping gas.
76. The process according to embodiment 75, wherein the stripping gas comprises molecular oxygen or is free of molecular oxygen.
77. The process according to any of embodiments 1 to 76, wherein the thermolysis of the poly-3-hydroxypropionate is effected in the presence of at least one polymerization inhibitor.
78. The process according to embodiment 77, wherein the thermolysis of the poly-3-hydroxypropionate, based on the weight of the mass of the poly-3-hydroxypropionate, is effected in the presence of 10 to 1000 ppm by weight of at least one polymerization inhibitor.
79. The process according to embodiment 77 or 78, wherein the at least one polymerization inhibitor is at least one polymerization inhibitor from the group consisting of o-, m- and p-cresol, 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, hydroquinone, catechol, resorcinol, 2-methylhydroquinone and 2,5-di-tert-butylhydroquinone, para-aminophenol, para-nitrosophenol, 2-methoxyphenol, 2-ethoxyphenol, 4-methoxyphenol, mono- and di-tert-butyl-4-methoxyphenol, α-tocopherol, 4-hydroxy-2,2,6,6-tetramethylpiperidine N-oxyl, 2,2,6,6-tetramethylpiperidine N-oxyl, 4,4′,4″-tris(2,2,6,6-tetramethylpiperidine N-oxyl) phosphite, 3-oxo-2,2,5,5-tetra-methylpyrrolidine N-oxyl, N,N-diphenylamine, N-nitrosodiphenylamine, N,N′-dialkyl-para-phenylenediamine, where the alkyl radicals may be the same or different and each independently consist of 1 to 4 carbon atoms and may be straight-chain or branched, N,N-diethylhydroxylamine, triphenylphosphine, triphenyl phosphite, hypophosphorous acid, triethyl phosphite, diphenyl sulfide, phenothiazine, and all aforementioned inhibitors optionally in combination with metal salts, for example the chlorides, dithiocarbonates, sulfates, salicylates or acetates of copper, manganese, cerium, nickel and/or chromium.
80. The process according to any of embodiments 1 to 79, wherein the poly-3-hydroxypropionate is at least one macromolecular compound having at least one structural section of the general formula I,

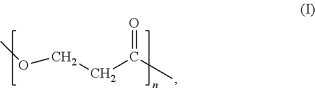

where n is an integer ≧6.
81. The process according to embodiment 80, wherein n≧8.
82. The process according to embodiment 80, wherein n≧10.
83. The process according to embodiment 80, wherein n≧15.
84. The process according to embodiment 80, wherein n≧20.
85. The process according to embodiment 80, wherein n≧25.
86. The process according to embodiment 80, wherein n≧30.
87. The process according to embodiment 80, wherein n≧40.
88. The process according to embodiment 80, wherein n≧50.
89. The process according to embodiment 80, wherein n≧60.
90. The process according to any of embodiments 80 to 89, wherein n≦30 000.
91. The process according to any of embodiments 80 to 90, wherein n≦25 000.
92. The process according to any of embodiments 80 to 91, wherein n≦20 000.
93. The process according to any of embodiments 80 to 92, wherein n≦15 000.
94. The process according to any of embodiments 80 to 93, wherein n≦10 000.
95. The process according to any of embodiments 80 to 94, wherein n≦8000.
96. The process according to any of embodiments 80 to 95, wherein n≦5000.
97. The process according to any of embodiments 80 to 96, wherein n≦2500.
98. The process according to any of embodiments 80 to 97, wherein n≦1500.
99. The process according to any of embodiments 80 to 98, wherein n≦1000.
100. The process according to any of embodiments 80 to 99, wherein n≦750.
101. The process according to any of embodiments 80 to 100, wherein n≦500.
102. The process according to any of embodiments 80 to 101, wherein n≦300.
103. The process according to any of embodiments 80 to 102, wherein n≦175.
104. The process according to any of embodiments 80 to 103, wherein n≦150.
105. The process according to any of embodiments 80 to 104, wherein n≦125.
106. The process according to any of embodiments 80 to 105, wherein n≦100.
107. The process according to any of embodiments 1 to 106, wherein the poly-3-hydroxypropionate is a copolymer or a homopolymer.
108. The process according to any of embodiments 80 to 107, wherein the proportion by weight of structural sections of the general formula (I) in the poly-3-hydroxypropionate is ≧40% by weight.
109. The process according to any of embodiments 80 to 107, wherein the proportion by weight of structural sections of the general formula (I) in the poly-3-hydroxypropionate is ≧50% by weight.
110. The process according to any of embodiments 80 to 107, wherein the proportion by weight of structural sections of the general formula (I) in the poly-3-hydroxypropionate is ≧60% by weight.
111. The process according to any of embodiments 80 to 107, wherein the proportion by weight of structural sections of the general formula (I) in the poly-3-hydroxypropionate is ≧70% by weight.
112. The process according to any of embodiments 80 to 107, wherein the proportion by weight of structural sections of the general formula (I) in the poly-3-hydroxypropionate is ≧80% by weight.
113. The process according to any of embodiments 80 to 107, wherein the proportion by weight of structural sections of the general formula (I) in the poly-3-hydroxypropionate is ≧90% by weight.
114. The process according to any of embodiments 80 to 107, wherein the proportion by weight of structural sections of the general formula (I) in the poly-3-hydroxypropionate is ≧95% by weight.
115. The process according to any of embodiments 80 to 107, wherein the proportion by weight of structural sections of the general formula (I) in the poly-3-hydroxypropionate is ≧98% by weight.
116. The process according to any of embodiments 80 to 107, wherein the proportion by weight of structural sections of the general formula (I) in the poly-3-hydroxypropionate is ≧99% by weight.
117. The process according to any of embodiments 1 to 116, wherein the poly-3-hydroxypropionate has been obtained by a dehydrating polycondensation of 3-hydroxypropionic acid, or by a process for ring-opening polymerization of β-propiolactone, or by a process for carbonylating reaction of ethylene oxide dissolved in a solvent with CO in the presence of at least one cobalt-comprising catalyst system, or by biotechnological means in a biological organism (for example from at least one sugar).
118. The process according to any of embodiments 1 to 117, wherein the polydispersity of the poly-3-hydroxypropionate is ≦2.5.
119. The process according to any of embodiments 1 to 118, wherein the weight-average relative molecular weight M_{W} of the poly-3-hydroxypropionate is 1000 to 2 000 000.
120. The process according to any of embodiments 1 to 119, wherein the poly-3-hydroxypropionate does not have a vinylic head group and/or a vinylic end group.
121. The process according to any of embodiments 1 to 120, wherein the acrylic acid is converted from the acrylic acid-comprising gas phase formed in the thermolysis of the poly-3-hydroxypropionate to the liquid phase by absorptive and/or condensative measures.
122. The process according to embodiment 121, wherein the acrylic acid is separated from the liquid phase in an elevated purity compared to the liquid phase using at least one thermal separation process, and the at least one thermal separation process comprises at least one rectification and/or crystallization of the acrylic acid present in the liquid phase.
123. The process according to embodiment 122, wherein the crystallization is a suspension crystallization to obtain a crystal suspension comprising acrylic acid crystals.
124. The process according to embodiment 123, which is followed by a separating process in which the acrylic acid crystals are separated from the crystal suspension in a wash melt wash column.
125. The process according to embodiment 124, wherein the wash melt wash column is a hydraulic wash melt wash column.
126. The process according to any of embodiments 1 to 125, wherein the process for preparing acrylic acid is followed by a process for free-radical polymerization in which the acrylic acid prepared is polymerized to polymer with free-radical initiation as such and/or in the form of the conjugate Brønsted base thereof, and optionally in a mixture with other mono- and/or polyunsaturated compounds.
127. The process according to any of embodiments 1 to 126, wherein the melting point of the poly-3-hydroxypropionate at a pressure of 1.0133·10⁵ Pa is ≦200° C.
128. The process according to any of embodiments 1 to 126, wherein the melting point of the poly-3-hydroxypropionate at a pressure of 1.0133·10⁵ Pa is ≦150° C.
129. The process according to any of embodiments 1 to 126, wherein the melting point of the poly-3-hydroxypropionate at a pressure of 1.0133·10⁵ Pa is ≦100° C.
130. The process according to any of embodiments 1 to 129, wherein the melting point of the poly-3-hydroxypropionate at a pressure of 1.0133·10⁵ Pa is ≧50° C.
131. The process according to any of embodiments 1 to 130, wherein the relative weight-average molecular weight of the poly-3-hydroxypropionate is 1000 to 1 000 000.
132. The process according to any of embodiments 1 to 131, wherein the relative weight-average molecular weight of the poly-3-hydroxypropionate is 1000 to 500 000.
133. The process according to any of embodiments 1 to 132, wherein the relative weight-average molecular weight of the poly-3-hydroxypropionate is 1000 to 400 000.
134. The process according to any of embodiments 1 to 133, wherein the relative weight-average molecular weight of the poly-3-hydroxypropionate is 1000 to 200 000.
135. The process according to any of embodiments 1 to 134, wherein the relative weight-average molecular weight of the poly-3-hydroxypropionate is 1000 to 100 000.
136. The process according to any of embodiments 1 to 135, wherein the relative weight-average molecular weight of the poly-3-hydroxypropionate is 1000 to 20 000.
137. The process according to any of embodiments 1 to 136, wherein the relative weight-average molecular weight of the poly-3-hydroxypropionate is 1000 to 15 000.
138. The process according to any of embodiments 1 to 137, wherein the relative weight-average molecular weight of the poly-3-hydroxypropionate is 2000 to 12 000.
139. The process according to any of embodiments 1 to 138, wherein the relative weight-average molecular weight of the poly-3-hydroxypropionate is 3000 to 10 000.
140. The process according to any of embodiments 1 to 139, wherein the relative weight-average molecular weight of the poly-3-hydroxypropionate is 5000 to 10 000.
141. The process according to any of embodiments 1 to 127, wherein the poly-3-hydroxypropionate has been obtained by biotechnological means (for example from at least one sugar), and the relative weight-average molecular weight thereof is ≦200 000.
142. The process according to embodiment 141, wherein the relative weight-average molecular weight thereof is ≦100 000.
143. The process according to embodiment 141 or 142, wherein the relative weight-average molecular weight is ≧1000.
144. The process according to embodiment 141 or 142, wherein the relative weight-average molecular weight is ≧5000.
145. The process according to any of embodiments 1 to 144, wherein the at least one molecular organic active compound has no aromatic (nor a heteroaromatic) ring system.
146. The process according to any of embodiments 1 to 144, wherein the at least one molecular organic active compound is pentamethyldiethylenetriamine.
147. The process according to any of embodiments 1 to 144, wherein the at least one molecular organic active compound is N,N,N′,N′-tetramethyl-1,6-hexanediamine.
148. The process according to any of embodiments 1 to 144, wherein the at least one molecular organic active compound is bis(2-dimethylaminoethyl)ether.
149. The process according to any of embodiments 1 to 144, wherein the at least one molecular organic active compound is 2,2′-dimorpholinodiethyl ether.
150. The process according to any of embodiments 1 to 144, wherein the at least one molecular organic active compound is N,N′-diethylethanolamine.
151. The process according to any of embodiments 1 to 144, wherein the at least one molecular organic active compound is N,N-dimethylcyclohexylamine.
152. The process according to any of embodiments 1 to 144, wherein the at least one molecular organic active compound is N-methylimidazole.
153. The process according to any of embodiments 1 to 144, wherein the at least one molecular organic active compound is 1,2-dimethylimidazole.
154. The process according to any of embodiments 1 to 153, wherein the polydispersity of the poly-3-hydroxypropionate is ≦2.0.
155. The process according to any of embodiments 1 to 153, wherein the polydispersity of the poly-3-hydroxypropionate is ≦1.5.
156. The process according to any of embodiments 1 to 153, wherein the polydispersity of the poly-3-hydroxypropionate is 1.2 to 2.0.
157. The process according to any of embodiments 1 to 153, wherein the polydispersity of the poly-3-hydroxypropionate is 1.5 to 1.8.
158. The process according to any of embodiments 77 to 157, wherein the thermolysis of the poly-3-hydroxypropionate, based on the weight of the mass of the poly-3-hydroxypropionate, is effected in the presence of 50 to 500 ppm by weight of at least one polymerization inhibitor.
159. The process according to any of embodiments 77 to 158, wherein the thermolysis of the poly-3-hydroxypropionate, based on the weight of the mass of the poly-3-hydroxypropionate, is effected in the presence of 150 to 350 ppm by weight of at least one polymerization inhibitor.

### EXAMPLES AND COMPARATIVE EXAMPLES

(Starting materials and analysis methods detailed and specified for the first time in each case for description of examples and comparative examples in the following experiments were used in a corresponding manner at the corresponding point in subsequent experiments, unless explicitly stated otherwise; all precipitations and washes of poly-3-hydroxypropionate prepared by carbonylation of ethylene oxide in the presence of a cobalt-comprising catalyst system were conducted under air)

A) Preparation of poly-3-hydroxypropionate
1. Preparation of poly-3-hydroxypropionate by carbonylation of ethylene oxide dissolved in diglyme with CO in the presence of a cobalt-comprising catalyst system (the preparation was based on the thesis “Multi-Site Catalysis—Novel Strategies to Biodegradable Polyesters from Epoxides/CO and Macrocyclic Complexes as Enzyme Models” by Markus Allmendinger, University of Ulm (2003), and on EP 577206 A2)

The carbonylating conversion was effected in an autoclave A stirrable with a paddle stirrer (the paddle stirrer was moved by means of magnetic coupling), the reaction space of which was optionally heatable or coolable from the outside. All surfaces in contact with the reaction space were manufactured from Hastelloy HC4. The reaction space of the autoclave had a circular cylindrical geometry. The height of the circular cylinder was 335 mm. The internal diameter of the circular cylinder was 107 mm. The shell of the reaction space had a wall thickness of 19 mm (Hastelloy HC4). The top of the autoclave was equipped with a gas inlet/gas outlet valve V which opened into the reaction space. The temperature in the reaction space was determined with the aid of a thermocouple. The reaction temperature was regulated under electronic control. The internal pressure in the reaction space was monitored continuously with an appropriate sensor.

The reaction space of the autoclave was at first inertized with argon (contents in the Ar: ≧99.999% by vol. of Ar, ≦2 ppm by vol. of O₂, ≦3 ppm by vol. of H₂O and ≦0.5 ppm by vol. of total amount of hydrocarbons).

Subsequently, the autoclave A at a controlled temperature of 10° C. was charged under argon with 16.0 g of dicobalt octacarbonyl (Co₂(CO)₈; supplier: Sigma-Aldrich; specification: 1-10% hexane, ≧90% Co, catalog number: 60811), 8.7 g of 3-hydroxypyridine (supplier: Sigma-Aldrich; specification: 99% content, catalog number: H57009) and 1001.2 g of diglyme (supplier: Sigma-Aldrich; specification: 99% content, catalog number: M1402), and the autoclave was subsequently closed. The temperature of the two solids was 25° C. and the temperature of the diglyme was 10° C. Then, while maintaining the internal temperature of 10° C., carbon monoxide was injected into the autoclave through the valve V until the pressure in the reaction space was 1.5·10⁶ Pa (carbon monoxide from BASF SE, specification: 99.2% CO). Subsequently, the temperature in the reaction space was increased to 28° C. in order to verify the integrity of the autoclave A (over a period of 50 min). Then the atmosphere in the reaction space was decompressed to an internal pressure of 10⁶ Pa by opening the valve V. The temperature of 28° C. in the interior was maintained.

Subsequently, while maintaining the internal temperature of 28° C., 97.8 g of ethylene oxide (1.5 g/min) were pumped through the valve V into the reaction space (supplier: BASF SE; specification: 99.9% purity). Thereafter, carbon monoxide was again injected into the autoclave until the pressure in the reaction space reached 6·10⁶ Pa (while maintaining the internal temperature of 28° C.).

Then, while stirring (700 rpm), the temperature in the reaction space of autoclave A was increased in an essentially linear manner to 75° C. within 45 min. This temperature was maintained while stirring for 8 h. The pressure in the reaction space fell to 3·10⁶ Pa within this period. Then the heating of autoclave A was switched off. Within 6 h, the temperature in the stirred reaction space cooled down in an essentially exponential manner to 25° C. (after 66 min the internal temperature had fallen to 60° C., after 165 min to 40° C. and after 255 min to 30° C.). The corresponding pressure in the reaction space was 2.8·10⁶ Pa. Then autoclave A was decompressed to standard pressure and the reaction space was purged with argon (10⁶ Pa) three times in succession.

In the reaction space were 1106.3 g of a dark red/brown solution as product mixture A.

Product mixture A was left to stand in a closed glass flask in a cooling space at a temperature of 7° C. for 12 h. The poly-3-hydroxypropionate which precipitated out was filtered off and the filtercake was washed with 300 g of methanol at a temperature of 25° C. The washed filtercake was dried for 10 h (10 hPa, 25° C.). The 41.4 g of poly-3-hydroxypropionate thus removed from product mixture A (first fraction) still comprised, based on the weight of the mass thereof, 1.6% by weight of cobalt (the starting weight content of Co in product mixture A, based on the weight of the maximum possible amount of poly-3-hydroxypropionate formed, was 2.97% by weight). The weight-average relative molecular weight was M_{W}=7220.

The filtrate obtained in the removal of the poly-3-hydroxypropionate by filtration was analyzed by gas chromatography. It comprised (reported as area percent of the total area of the GC peaks) 0.9% ethylene oxide, 92.7% diglyme, 1.0% of the β-propiolactone by-product and 0.6% of the succinic anhydride by-product.

The material was combined with the methanol which had been sucked through in the course of washing of the poly-3-hydroxypropionate which had been filtered off (first fraction). The mixture thus obtained was left to stand in a cooling space at a temperature of 7° C. for 12 h. The poly-3-hydroxypropionate which precipitated out was filtered off again and the resulting filtercake was washed with 300 g of methanol at a temperature of 25° C. (as always, the methanol was sucked through the filtercake). The washed filtercake was dried again at 10 hPa and 25° C. for 10 h.

The mass of the poly-3-hydroxypropionate separated in this way from product mixture A as the second fraction was 88.0 g. Based on the weight of the mass thereof, it still comprised 1.6% by weight of cobalt. The weight-average relative molecular weight M_{W} thereof was 5640.

The filtrate obtained in the removal of the second fraction of poly-3-hydroxypropionate by filtration was combined with the methanol sucked through in the course of washing of the second fraction of poly-3-hydroxypropionate. The mixture thus obtained was left to stand in a cooling space at a temperature of 7° C. for 12 h. The poly-3-hydroxypropionate obtained was filtered off again (third fraction) and the resulting filtercake was washed with 300 g of methanol at a temperature of 25° C. The washed filtercake was dried again at 10 hPa and 25° C. for 10 h.

The mass of the poly-3-hydroxypropionate removed in this way as the third fraction from product mixture A was 5.8 g. Based on the weight of the mass thereof, it still comprised 1.8% by weight of cobalt. The weight-average relative molecular weight M_{W} thereof was 5240.

The filtrate obtained in the removal of the third fraction of poly-3-hydroxypropionate by filtration was combined with the methanol sucked through in the course of washing of the third fraction of poly-3-hydroxypropionate. The resulting mixture was left to stand in a cooling space at a temperature of 7° C. for 12 h. The poly-3-hydroxypropionate which precipitated out was filtered off again (fourth fraction) and the resulting filtercake was washed with 300 g of methanol at a temperature of 25° C. The washed filtercake was dried again at 10 hPa and 25° C. for 10 h.

The mass of the poly-3-hydroxypropionate thus removed from product mixture A as the third fraction was 5.3 g. Based on the weight of the mass thereof, it comprised 2.7% by weight of cobalt. The weight-average relative molecular weight M_{W} thereof was 4230.

The elevated cobalt content of the third fraction is attributed to the fact that cobalt which was previously still dissolved now apparently also precipitates as a separate cobalt salt in the resulting solvent mixture.

A total of 140.2 g of poly-3-hydroxypropionate are removed from product mixture A. This is 87.6% of the theoretically possible maximum yield.

The cobalt contents were determined by inductively coupled plasma optical ion emission spectroscopy (ICP-OES).

The instrument used was a varian 720-ES ICP-OES spectrometer. The wavelength of the spectral line of Co used for analysis was 237.86 nm.

For sample preparation, 0.1 g of the sample to be analyzed in each case was converted to ash with a mixture of concentrated sulfuric acid, concentrated nitric acid and concentrated perchloric acid (as strongly oxidizing acids) in a quartz test tube (using temperatures of up to 320° C., the acids were quantitatively fumed off). The remaining residue was taken up in concentrated hydrochloric acid and dissolved with heating and addition of water. The resulting solution was subsequently analyzed.

The molecular weights were determined by size exclusion chromatography (SEC/GPC). The elution curve was converted to the actual distribution curve with the aid of polymethyl methacrylate (PMMA) calibration curves. The calibration was effected with narrow-distribution PMMA standards, the relative molecular weights of which were within the range from M=800 to M=1 820 000. The values outside this elution range were extrapolated.

This experiment “A)1.” was repeated several times, and mixing of various fractions removed gave a poly-3-hydroxypropionate which, based on the weight of its mass, still comprised 2% by weight of Co.
2. Reduction of the cobalt content of the poly-3-hydroxypropionate comprising 2% by weight of Co from experiment “A)1.”

An 80 g sample of this poly-3-hydroxypropionate was washed with 658 g of a 12.5% by weight solution of acetic acid in water (the temperature of the acetic acid solution was 25° C.; it was sucked through the P3HP).

It was subsequently washed with 200 g of water (temperature=25° C.) and then with 200 g of methanol (temperature=25° C.), and the remaining solids were dried at 10 hPa and 25° C. for 10 h.

The cobalt content of the poly-3-hydroxypropionate thus obtained was 0.2% by weight.

The weight-average molecular weight prior to the wash was M_{W}=5930, and after the wash M_{W}=5810.

Analysis of the melting characteristics (this was effected by the method of dynamic differential calorimetry (DSC) on a Q2000 differential calorimeter from TA (Thermal Analysis) Instruments; the amount of sample was 8.2 mg each time and the heating/cooling rate was 20 K/min) gave a melting range of 65.7° C. to 79° C. for the P3HP prior to the wash, and of 65.4° C. to 71.6° C. after the wash.

The elemental analysis of the P3HP (which was effected on the basis of the full combustion of the respective sample with subsequent gas chromatography analysis of the combustion products using a CHN analyzer from Elementar Analysensysteme GmbH of the vario EL cube type, and using an O analyzer from EuroVektor of the EA type) gave (figures in % by weight):

### C: 47.8%;

### O: 42.6%;

### H: 5.6%; and

### N: 0.5%.

After the wash, the corresponding elemental analysis gave:

### C: 49.3%;

### O: 43.5%;

### H: 5.7%; and

### N: <0.5%.

Structure and end group analysis by means of MALDI-MS and GPC-MS (as described below) gave the following assignments for the washed P3HP:

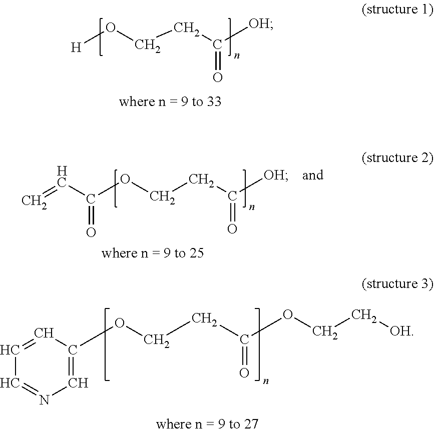

Quantitative determinations of the above structures were effected by the ¹H NMR method as described below.

The result was that the analyzed sample consisted to an extent of 99% of structure 1. Protons of the vinyl group in structure 2 were visible by their ¹H NMR signal. As were the protons of the ethylene glycol end groups. ¹H NMR signals of aromatic protons of structure 3 were undetectable.

To determine the end groups present and the structure of the solids removed, these were analyzed both by mass spectrometry with matrix-assisted laser desorption/ionization (MALDI-MS) and by gel permeation chromatography-mass spectrometry (GPC-MS).

For the MALDI-MS analysis, the sample to be analyzed was first dissolved completely in aqueous acetonitrile (50% by volume of water, 50% by volume of acetonitrile) and then applied to a MALDI steel target with 2,5-dihydroxybenzoic acid and sodium trifluoroacetate as matrix substances (both likewise dissolved in aqueous acetonitrile), and the solvent was removed. A nitrogen laser (pulse time 3 ns, wavelength=337 nm) was used to vaporize and ionize the analyte from the steel target in a mixture with the matrix.

The GPC-MS analysis proceeded from an extract of the sample to be analyzed in tetrahydrofuran (THF) (the sample did not dissolve fully in THF), the dissolved constituents of which were separated by means of GPC prior to the MS analysis thereof. The ionization was effected by means of electrospray ionization (ESI).

Quantitative determinations of the above structures were effected by means of ¹H NMR spectroscopy on a Bruker DPX 400/1 FT-NMR spectrometer at a ¹H carrier frequency of 400 MHZ.

The sample concentration was 5 mg of poly-3-hydroxypropionate dissolved in 1 ml of CDCl₃. The width of the excitation pulse was 8012.82 Hz. The sample temperature in the course of recording of the spectra was always 26.8° C. For excitation, a sequence of 30° pulses was used. 32 individual recordings in each case were accumulated to give the resulting spectrum.
3. Preparation of poly-3-hydroxypropionate by ring-opening polymerization of β-propiolactone (the synthesis was based on U.S. Pat. No. 4,357,462 A and on “Die Polymerisation von Lactonen, Teil 1: Homopolymerisation 4-, 6- and 7-gliedriger Lactone mit kationischen Initiatoren” in “Die Makromolekulare Chemie—New York—Hüthig & Wepf Verlag, Vol. 56, 1962, pages 179 ft”)

1 ml of boron trifluoride etherate (=catalyst; BF₃×(CH₃—CH₂—O—CH₂—CH₃)₂; supplier: Fluka; specification: purum, catalog number: 15719) was dissolved in 300 ml of methylene chloride (=solvent; supplier: BASF SE; specification: purity 98-100%) which had been stored over molecular sieve (3 Å) as a desiccant (n a glass 3-neck flask with capacity 750 ml, magnetic stirring was effected, the internal temperature was 20° C.).

A silicone oil bath was used to bring the solution to boiling (at standard pressure). Subsequently, 24.9 g of β-propiolactone (supplier: Alfa Aesar; specification: 97%, catalog number: B23197, LOT 10140573) were continuously added dropwise to the solution boiling under reflux within 20 min while stirring.

After addition had ended, the reaction mixture was kept under reflux for another 8 h while stirring. During the progressing reaction, the solution changed color from colorless through yellow to orange.

Thereafter, the solvent was removed by distillation while stirring under reduced pressure and at an oil bath temperature of 65° C. within 30 min.

There remained 27.2 g of an orange oil which was cooled to 25° C. and solidified in the manner of a wax at this temperature. To remove the catalyst system, 400 ml of methanol (25° C.) were added, the temperature of the mixture was warmed to 50° C. and the mixture was stirred at this temperature for 1 h and 50 min until the solids had dissolved completely. Then the solution was cooled again to 25° C., and a colorless solid precipitated out.

This was filtered off and the filtercake was washed twice in succession with 10 ml of methanol each time (the temperature of the methanol was 25° C.; the methanol was sucked through the filtercake) and then dried at 25° C. and 10 hPa for 8 h. There remained 12.4 g of a colorless powder. The weight-average relative molecular weight M_{W} thereof was 3000, with a polydispersity Q of 1.4.

The corresponding ¹H and ¹³C NMR spectra and the ATR-FT-IR spectrum corresponded to poly-3-hydroxypropionate having a purity of >95% by weight.

The ¹H and ¹³C NMR spectra were recorded on a Bruker DRX 500 FT-NMR spectrometer on solutions of poly-3-hydroxypropionate in CDCl₃. The magnetic field strength corresponded to a ¹H carrier frequency of 500 MHz.

The ATR infrared spectra were recorded with a Bruker Vertex 70 spectrometer with ATR (“attenuated total reflection”) and the method of FT-IR spectroscopy. The solid poly-3-hydroxypropionate was analyzed. For this purpose, the samples were additionally dried at 60° C. and 10 hPa for 12 h and then finely pulverized to enable optimal contact with the ATR crystal (in which total reflection proceeded).

B) Thermolytic Splittings of poly-3-hydroxypropionate Prepared in Experiments “A)1. to A)3.”
1. Noncatalyzed thermolysis of the poly-3-hydroxypropionate (P3HP) from experiment “A)3.” (comparative example 1)
a) The splitting apparatus manufactured from glass consisted of a round-bottom splitting flask (capacity 25 ml, three necks), atop which was a distillation system with thermometer, Liebig condenser, a product flask (capacity 10 ml, one neck) and a hose connection for offgas, which was open to the atmosphere.
   3.0 g of the poly-3-hydroxypropionate from experiment “A)3.” were weighed into the round-bottom splitting flask. Through the second neck of the splitting flask, a stream of molecular nitrogen (≧99.9% by vol. of N2; flow rate: 1.4 l/h; temperature: 25° C.) was supplied thereto as stripping gas over the full course of the thermolysis. This flowed through the splitting apparatus and left it again as part of the offgas which was conducted out of it through a cold trap, the temperature of which was kept at −78° C., via the offgas hose. The splitting flask filled with the P3HP was lowered down to the middle neck into a silicone oil bath preheated to 180° C. and heated by the oil bath at a working pressure of 1.0133·10⁵ Pa (standard pressure). A magnetic stirrer was used to stir the contents of the splitting flask.
   As the temperature in the splitting flask reached 60° C., the P3HP began to melt.
   As the internal temperature reached 80° C., the poly-3-hydroxypropionate had completely melted.
   On attainment of the internal temperature of 175° C., this was maintained while stirring for 300 min.
   The Liebig condenser was cooled in countercurrent with water which had an inflow temperature of 20° C.
   Condensable splitting products transported by the nitrogen steam were condensed in the Liebig condenser and the condensate was collected in the product flask which was likewise kept at a temperature of 20° C.
   Within the aforementioned 300 min, no condensate was obtained in the product flask.
b) A sample of 34.86 mg of the poly-3-hydroxypropionate from experiment “A)3.” was weighed into an Al₂O₃ crucible and the behavior thereof with increasing temperature was analyzed simultaneously by the method of thermogravimetry and by the method of dynamic differential calorimetry (“simultaneous TG-DSC analysis”).
   The analysis was effected with a “NETZSCH STA 449 F3 Jupiter®” thermal analysis apparatus from Netzsch Gerätebau GmbH.
   By means of FT-IR spectroscopy, the splitting gas formed in the thermolysis accompanying the thermal analysis was analyzed with respect to its main components.
   In the course of the analysis, the sample was first heated to 35° C. for 10 min and then the sample temperature was increased to 610° C. at a constant rate of 5 K/min under an argon stream (40 ml/min).
   As a function of temperature, the sample mass and the heat flow through the sample were detected (i.e. the dynamic differential calorimetry was executed in the form of dynamic heat flow differential calorimetry).
   The thermogram obtained, with reference to FT-IR spectroscopy, showed the following three endothermic processes:
      1. The melting of the P3HP without loss of mass;
         onset temperature (oT_{S}): 70.1° C.;
         peak temperature (pT_{S}): 93.6° C.
         oT_{S}=the temperature at which the melting of the sample verifiably sets in;
         pT_{S}=the temperature at which the melting operation has its highest rate;
      2. Thermolysis of the sample to acrylic acid;
         onset temperature (oT_{T}): 286.5° C.;
         peak temperature (pT_{T}): 340.0° C.;
         oT_{T}=the temperature at which the thermolysis verifiably sets in;
         pT_{T}=the temperature at which the thermolysis has its maximum splitting rate;
         Loss of mass: 98.8% of the starting mass;
         the splitting gas comprised, as main components, acrylic acid and traces of CO₂.
      3. Decomposition of the residual mass above 400° C.;
         no onset or peak temperature determinable since the end of the measurement range was attained at 610° C.;
         loss of mass by the end of the measurement range: 0.5% of the starting mass.
2. Thermolysis of the poly-3-hydroxypropionate (P3HP) from experiment “A)3.” in the presence of 3-hydroxypyridine as a splitting catalyst (comparative example 2)
   The procedure was as in experiment “B)1.a)”, except that the melting of the P3HP was followed by addition of 97 mg of 3-hydroxypyridine to the melt. As early as 15 min after attainment of the internal temperature of 175° C. in the splitting flask, the first condensate was obtained in the product flask (the product flask in this experiment “B)2.” and in all subsequent thermolysis experiments did not comprise any added polymerization inhibitor). After a total of 90 min at internal temperature 175° C., the residual melt still present in the splitting flask solidified. Thereafter, the splitting experiment was stopped. Condensate droplets adhering in the distillation system were vaporized by heating them with a hot air gun, liquefied in the Liebig condenser and collected in the product flask.
   The amount of condensate present in the product flask was 2.48 g.
   According to gas chromatography analysis, the condensate (based on the weight thereof) comprised 95.5% by weight of acrylic acid, 3.6% by weight of diacrylic acid (Michael adduct) and 0.8% by weight of higher Michael adducts of acrylic acid onto itself.
   Aldehydes were undetectable in the condensate. The condensate did not comprise any 3-hydroxypyridine.
   The mass of the remaining light brown, tacky residue in the splitting flask was 330 mg (11% by weight of the use amount of P3HP).
   The Michael adducts also stripped by the stripping gas can be retained in a simple manner here (and in all subsequent cases) if required by conducting the stream through a rectification column (for example a Vigreux column) operated under reflux to the product flask. The splitting yield of acrylic acid can be increased correspondingly.
3. Thermolysis of the poly-3-hydroxypropionate (P3HP) from experiment “A)3.” in the presence of pentamethylethylenetriamine (Lupragen® N301) as a splitting catalyst (example 1)
   a) The procedure was as in experiment “B)1.a)”, except that the melting of the P3HP was followed by addition of 87 mg of pentamethylethylenetriamine (supplier: BASF SE; specification: >98%, trade name: Lupragen® N301) to the melt. As early as 15 min after attainment of the internal temperature of 175° C. in the splitting flask, the first condensate was obtained in the product flask. After a total of 120 min at internal temperature 175° C., the residual melt still present in the splitting flask solidified (in tacky solid form). Thereafter, the splitting experiment was stopped. Condensate droplets adhering in the distillation system were vaporized by heating them with a hot air gun, liquefied in the Liebig condenser and collected in the product flask.
   The amount of condensate present in the product flask was 2.71 g.
   The condensate comprised 95.7% by weight of acrylic acid, 3.3% by weight of diacrylic acid (Michael adduct) and 0.5% by weight of higher Michael adducts of acrylic acid onto itself. Aldehydes were undetectable in the condensate. The condensate did not comprise any pentamethylethylenetriamine. The mass of the pale brown tacky residue remaining in the splitting flask was 150 mg (5% by weight of the use amount of P3HP).
   b) The procedure was as in experiment “B)1.b)”, except that the amount of P3HP sample was 36.65 mg and 0.68% by weight of pentamethylenetriamine had been added to this sample prior to the thermal analysis, based on the weight thereof.
   The resulting thermogram showed, with reference to FT-IR spectroscopy, the following three endothermic processes:
      1. The melting of the P3HP without loss of mass;
         onset temperature: 69.6° C.;
         onset temperature: 93.3° C.
      2. Thermolysis of the sample to acrylic acid;
         onset temperature: 208.7° C.;
         peak temperature: 259.7° C.;
         loss of mass: 98.9% of the starting mass;
         the splitting gas comprises acrylic acid as the main component and traces of CO₂.
      3. Decomposition of the residual mass above 300° C.;
         no onset and peak temperatures determinable;
         loss of mass up to the end of the measurement range: 0.3% of the starting mass.
4. Thermolysis of the poly-3-hydroxypropionate (P3HP) from experiment “A)3.” in the presence of N-benzylamine as a splitting catalyst (comparative example 3)
   The procedure was as in experiment “B)1.a)”, except that, after the melting of the P3HP, 90 mg of N-benzylamine (supplier: Sigma-Aldrich; specification: >99%, catalog number: 185701) were added to the melt. On attainment of the internal temperature of 175° C., this was maintained with stirring for another 300 min. Then the thermolysis experiment was stopped.
   Within the aforementioned 300 min, no condensate was obtained in the product flask.
   The contents remaining in the splitting flask solidified at an internal temperature of 55° C. to give a pale beige wax. The amount of the wax was 3.06 g (99.0% by weight of the use amount of P3HP and benzylamine). The weight-average relative molecular weight M_{W} of the P3HP content was 1900 after the experiment, at a polydispersity Q of 2.7.
5. Simulation of a thermolysis of poly-3-hydroxypropionate (P3HP) present in biomass in the presence of pentamethylethylenetriamine as a splitting catalyst (Example 2)

This experiment simulates an inventive thermolysis from dried bacterial biomass, the bacteria of which have formed poly-3-hydroxypropionate and the cell walls of which have been destroyed, in order to improve access of the splitting catalyst to the poly-3-hydroxypropionate.

The procedure was essentially as in experiment “B)1.a)”. Instead of using only 3 g of poly-3-hydroxypropionate from experiment “A)3.”, however, 3 g of a mixture of 2.4 g of the poly-3-hydroxypropionate from experiment “A)3.” (P3HP) and 0.6 g of dry biomass (mass ratio of “dry biomass: P3HP=1:4”, as is typical according to WO 2011/100608 A1 for P3HP prepared by biotechnological means from glucose in appropriately modified bacteria) were prepared and finely mixed by trituration in a mortar (the biomass comprised autoclaved (15 min at 121° C. and 2. 105 Pa of steam) and freeze-dried bacteria of the E. coli strain JM 109 type). The resulting mixture was used in its entirety as the sample to be split. The remaining procedure was at first as in experiment “B)1.a)”. An internal temperature of approx. 175° C. was established within 10 min, in the course of which the flask contents did not liquefy. Within 30 min, no distillate was obtained in the product flask, and therefore 90.0 mg of pentamethylethylenetriamine were added to the splitting flask. After a further 15 min, again no distillate was obtained, and so the bath temperature was increased. 15 min after attainment of an internal temperature of 185° C., the first distillate was finally collected and, after a total of 120 min at this temperature, the splitting was ended since no further distillate distilled over. Distillate droplets remaining in the distillation system were vaporized by heating with a hot air gun, liquefied in the Liebig condenser and collected in the distillate flask. The amount of condensate present in the product flask was 2.01 g.

The condensate comprised 97.1% by weight of acrylic acid, 2.1% by weight of diacrylic acid (Michael adduct) and 0.5% by weight of higher Michael adducts of acrylic acid onto itself.

Aldehydes were undetectable in the condensate. The condensate did not comprise any pentamethylethylenetriamine. The condensate likewise did not comprise any detectable amounts of constituents that can be traced back to the biomass. In the splitting flask remained 800 mg (26.7% by weight based on the total amount of biomass and poly-3-hydroxypropionate weighed in) of a light brown, tacky residue. If the 600 mg starting weight of biomass is deducted from the calculation, 8.3% by weight based on P3HP therein was still present in the splitting flask).
6. Thermolysis of the poly-3-hydroxypropionate (P3HP) still comprising 2% by weight of cobalt from experiment “A)1.” (comparative example 4)
   a) The procedure was as in experiment “B)1.a)”, except that 3.0 g of the poly-3-hydroxypropionate comprising 2% by weight of Co from experiment “A)1.” were weighed into the splitting flask. 30 minutes after attainment of the internal temperature of 175° C. in the splitting flask, the first condensate was obtained in the product flask. After a total of 90 minutes at internal temperature 175° C., the residual melt still present in the splitting flask became extremely viscous, and so the splitting test was stopped. Condensate droplets adhering in the distillation system were vaporized by heating them with a hot air gun, liquefied in the Liebig condenser and collected in the product flask.
      The amount of condensate present in the product flask was 2.14 g (compounds which acted as splitting catalysts were, for example, those of the structure 3, as detected in experiment “A)2.”).
      The condensate comprised 95.3% by weight of acrylic acid, 3.7% by weight of diacrylic acid (Michael adduct) and 0.5% by weight of higher Michael adducts of acrylic acid onto itself. Aldehydes were undetectable in the condensate.
      The mass of the dark brown residue which was glassy and brittle at 25° C. and remained in the splitting flask was 710 mg (24% by weight of the use amount of P3HP).
      An elemental analysis of the splitting residue gave the following contents based on the weight of the mass thereof: 12% by weight of Co, 46.6% by weight of C, 4.5% by weight of H, 2.9% by weight of N and 34% by weight of O.
      This result correlates with a substance mixture composed of 12% by weight of Co, 19.7% by weight of 3-hydroxypyridine and 68.3% by weight of a substance having the elemental composition 50.1% by weight of C, 5.1% by weight of H and 44.9% by weight of O. The latter corresponds satisfactorily to the theoretical elemental composition of P3HP: 50.0% by weight of C, 5.59% by weight of H and 44.4% by weight of O.
   b) The procedure was as in experiment “B)1.b)”, except that the sample analyzed was 37.70 mg of the poly-3-hydroxypropionate comprising 2% by weight of Co from experiment “A)1.”.
      The resulting thermogram showed, with reference to FT-IR spectroscopy, the following three endothermic processes:
      1. The melting of the P3HP (with a loss of mass of 0.4% of the starting mass);
         onset temperature: 62.9° C.;
         peak temperature: 76.0° C.
      2. Thermolysis of the sample to acrylic acid;
         onset temperature: 204.3° C.;
         peak temperature: 235.1° C.;
         loss of mass: 86.0% of the starting mass;
         the splitting gas comprised acrylic acid as the main component and traces of CO₂ and methane.
      3. Decomposition of the residual mass above 300° C.;
         no onset or peak temperature determinable;
         loss of mass up to the end of the measurement range: 4.7% of the starting mass.
7. Thermolysis of the poly-3-hydroxypropionate (P3HP) still comprising 2% by weight of Co from experiment “A)1.” in the additional presence of pentamethylethylenetriamine as a splitting catalyst (example 3)
   a) The procedure was as in experiment “6.a)”, except that, in addition to the 3.0 g of the poly-3-hydroxypropionate comprising 2% by weight of Co, after the melting thereof, 87 mg of pentamethylethylenetriamine were also added to the splitting flask. As early as 15 min after attainment of the internal temperature of 175° C. in the splitting flask, the first condensate was obtained in the product flask. After a total of 90 min at internal temperature 175° C., the residual melt still present in the splitting flask became distinctly viscous, and so the splitting experiment was stopped. Condensate droplets adhering in the distillation system were vaporized by heating thereof with a hot air gun, liquefied in the Liebig condenser and collected in the product flask.
      The amount of condensate present in the product flask was 2.21 g. The condensate comprised 96.1% by weight of acrylic acid, 3.2% by weight of diacrylic acid (Michael adduct) and 0.6% by weight of higher Michael adducts of acrylic acid onto itself. Aldehydes were undetectable in the condensate. The condensate did not comprise any pentamethylethylenetriamine.
      The mass of the dark brown residue which was glassy and brittle at 25° C. and remained in the splitting flask was 690 mg (23% by weight of the use amount of P3HP). In other words, the pentamethylethylenetriamine added as a splitting catalyst is unable to significantly reduce the splitting residue in the presence of Co compared to experiment “6.a)”.
   b) The procedure was as in experiment “6.b)”, except that the sample of P3HP was 35.43 mg of the poly-3-hydroxypropionate comprising 2% by weight of Co, and 0.58% by weight of pentamethylethylenetriamine, based on the weight thereof, was added to this sample prior to the thermal analysis.
      The resulting thermogram showed, with reference to FT-IR spectroscopy, the following three endothermic processes:
      1. The melting of the P3HP (with a loss of mass of 0.4% of the starting mass);
         onset temperature: 62.6° C.;
         peak temperature: 75.5° C.
      2. Thermolysis of the sample to acrylic acid;
         onset temperature: 191.5° C.;
         peak temperature: 222.6° C.;
         loss of mass: 88.4% of the starting mass;
         the splitting gas comprised acrylic acid as the main component and traces of CO₂ and methane.
      3. Decomposition of the residual mass above 290° C.;
         no onset and peak temperature was determinable;
         loss of mass up to the end of the measurement range: 4.6% of the starting mass.
         In other words, the added pentamethylethylenetriamine considerably lowers the activation energy required for the thermolysis compared to experiment “6.b)” in spite of the cobalt content.
8. Thermolysis of the poly-3-hydroxypropionate (P3HP) comprising only 0.2% by weight of Co from experiment “A)2.” (comparative example 5)
   a) The procedure was as in experiment “B)1.a)”, except that 3.0 g of the poly-3-hydroxypropionate comprising 0.2% by weight of Co from example 2 were weighed into the splitting flask.
      30 minutes after attainment of the internal temperature of 175° C. in the splitting flask, the first condensate was obtained in the product flask. After a total of 135 min at internal temperature 175° C., the residual melt still present in the splitting flask became distinctly viscous, and so the splitting experiment was stopped. Condensate droplets adhering in the distillation system were vaporized by heating them with a hot air gun, liquefied in the Liebig condenser and collected in the product flask.
      The amount of condensate present in the product flask was 2.51 g (compounds which acted as splitting catalysts were, for example, those of the structure 3, as detected in experiment “A)2.”). The condensate comprised 95.6% by weight of acrylic acid, 3.2% by weight of diacrylic acid (Michael adduct) and 0.6% by weight of higher Michael adducts of acrylic acid onto itself. Aldehydes were undetectable in the condensate.
      The mass of the dark brown residue which was glassy and brittle at 25° C. and remained in the splitting flask was 360 mg (12% by weight of the use amount of P3HP).
   b) The procedure was as in experiment “B)1.b)”, except that the sample analyzed was 36.65 mg of the poly-3-hydroxypropionate comprising 0.2% by weight of cobalt from example 2.
      The resulting thermogram showed, with reference to FT-IR spectroscopy, the following three endothermic processes:
      1. The melting of the P3HP without loss of mass;
      onset temperature: 60.9° C.;
      peak temperature: 86.9° C.
      2. Thermolysis of the sample to acrylic acid;
         onset temperature: 197.2° C.;
         peak temperature: 236.4° C.;
         loss of mass: 97.3% of the starting mass;
         the splitting gas comprised acrylic acid as the main component and traces of CO₂.
      3. Decomposition of the residual mass above 290° C.;
         no onset and peak temperatures determinable;
         loss of mass up to the end of the measurement range: 1.0% of the starting mass.
9. Thermolysis of the poly-3-hydroxypropionate (P3HP) comprising only 0.2% by weight of Co from experiment “A)2.” in the additional presence of pentamethylethylenetriamine as a splitting catalyst (example 4)
   a) The procedure was as in experiment “8.a)”, except that, in addition to the 3.0 g of the poly-3-hydroxypropionate comprising 0.2% by weight of Co, after the melting thereof, 87 g of pentamethylethylenetriamine were also added to the splitting flask. As early as 15 min after attainment of the internal temperature of 175° C. in the splitting flask, the first condensate was obtained in the product flask. After a total of 90 min at internal temperature 175° C., the residual melt still present in the splitting flask became distinctly viscous, and so the splitting experiment was stopped. Condensate droplets adhering in the distillation system were vaporized by heating thereof with a hot air gun, liquefied in the Liebig condenser and collected in the product flask.
      The amount of condensate present in the product flask was 2.56 g. The condensate comprised 96.2% by weight of acrylic acid, 2.9% by weight of diacrylic acid (Michael adduct) and 0.5% by weight of higher Michael adducts of acrylic acid onto itself. Aldehydes were undetectable in the condensate. The condensate did not comprise any pentamethylethylenetriamine.
      The mass of the dark brown residue which was glassy and brittle at 25° C. and remained in the splitting flask was 240 mg (8% by weight of the use amount of P3HP).
   b) The procedure was as in experiment “8.b)”, except that the sample of P3HP was 35.02 mg of the poly-3-hydroxypropionate comprising 0.2% by weight of cobalt from experiment “A)2.”, and 0.56% by weight of pentamethylethylenetriamine, based on the weight thereof, was added to this sample prior to the thermal analysis. The resulting thermogram showed, with reference to FT-IR spectroscopy, the following three endothermic processes:
      1. The melting of the P3HP without loss of mass;
         onset temperature: 60.6° C.;
         peak temperature: 84.8° C.
      2. Thermolysis of the sample to acrylic acid;
         onset temperature: 192.9° C.;
         peak temperature: 228.3° C.;
         loss of mass: 97.4% of the starting mass;
         the splitting gas comprised acrylic acid as the main component and traces of CO₂.
      3. Decomposition of the residual mass above 290° C.;
         no onset and peak temperatures determinable;
         loss of mass up to the end of the measurement range: 1.2% of the starting mass.
10. Thermolysis of a mixture of two poly-3-hydroxypropionates (P3HP): the P3HP from experiment “A)3.” and the P3HP comprising 2% by weight of cobalt based on the weight of the mass thereof from experiment “A)1.” (comparative example 6)
   The procedure was as in experiment “B1.a)”, except that a mixture of 2.5 g of the P3HP from experiment “A)3” and 2.5 g of the P3HP comprising 2% by weight of Co based on the weight of the mass thereof from experiment “A)1” was weighed in. 30 minutes after attainment of the internal temperature of 175° C. in the splitting flask, the first condensate was obtained in the product flask. After a total of 120 min at an internal temperature of 175° C., the residual melt still present in the splitting flask became distinctly viscous, and so the splitting experiment was stopped. Condensate droplets adhering in the distillation system were vaporized by heating them with a hot air gun, liquefied in Liebig condenser and collected in the product flask.
   The amount of condensate present in the product flask was 4.15 g. The condensate comprised 96.8% by weight of acrylic acid, 2.7% by weight of diacrylic acid (Michael adduct) and 0.3% by weight of higher Michael adducts of acrylic acid onto itself. Aldehydes were undetectable in the condensate.
   The mass of the dark brown residue which was glassy and brittle at 25° C. and remained in the splitting flask was 580 mg (12% by weight of the use amount of P3HP).
   The experiment shows that compounds of structure 3, for example, present in the P3HP from experiment “A)1.”, as detected in experiment “A)2.”, can act as regular splitting catalysts.
11. Evidence of the removability of pentamethylethylenetriamine used as a splitting catalyst from the splitting residue of experiment “B)3.a)” by gas chromatography separation of constituents which escape in gaseous form in the course of thermal treatment of this splitting residue and subsequent elucidation of the structure of these constituents by mass spectrometry (method of programmed pyrolysis GC/MS coupling) and FT-IR
   The thermal treatment of the splitting residue was effected in a circular cylindrical crucible made of V2A steel (height: 6.2 mm; wall thickness: 0.2 mm; external diameter: 2.5 mm). The sample of splitting residue from experiment “B)3.a)” weighed into the crucible was 0.23 mg. The crucible was introduced into the center of a circular cylindrical tube made of quartz glass (height 25 mm; internal diameter 5 mm; wall thickness 0.5 mm). The quartz glass tube was electrically heatable from the outside.
   A gas stream of He was conducted through the quartz glass tube (20 ml/min, inlet temperature into the quartz glass tube=25° C.), and this flowed in the direction of the crucible present in the tube (the opening of the crucible faced in the direction of the He stream), took up any gaseous constituents escaping therefrom and conveyed them in flow direction into a gas chromatography separating column. The length of the separating column was 30 m; the internal diameter thereof was 0.25 mm. As a stationary phase, it had a film of polydimethylsiloxane of layer thickness 1 μm (this column had been purchased commercially from Agilent Technologies as the “HP-1 ms” model).
   The start temperature of the electrical heating of the quartz tube was 100° C. This was increased to 400° C. with a ramp of 10° C./min and then held at this temperature.
   Until the attainment of 400° C., the constituents which exited in gaseous form from the sample thermally treated in the crucible and were conveyed with the He stream in the separating column were cryofocused on entry into it. For this purpose, the entire separating column was within a Dewar vessel filled with liquid nitrogen.
   Subsequently, the temperature of the entire separating column was increased to 40° C. and held at this temperature for 2 min. Then the temperature of the entire column was increased at a heating rate of 6° C./min up to a final temperature of 320° C. Finally, this final temperature was maintained for another 13 min. Over the entire period, the He stream flowed through the heated quartz glass tube comprising the crucible into the separating column and from the separating column into a mass spectrometer.
   In addition, in a further experiment, the gas stream flowing out of the separating column was analyzed by means of FT-IR.
   Pentamethylethylenetriamine was identified unambiguously as the main constituent in the He stream.

U.S. Provisional Patent Application No. 61/671,823, filed Jul. 16, 2012, is incorporated into the present application by literature reference. With regard to the abovementioned teachings, numerous changes and deviations from the present invention are possible. It can therefore be assumed that the invention, within the scope of the appended claims, can be performed differently than the way described specifically herein.

1. A process for preparing acrylic acid, comprising:
thermolyzing poly-3-hydroxypropionate in the presence of a catalyst of at least one molecular organic active compound comprising a tertiary nitrogen atom covalently bonded to three different carbon atoms, thereby obtaining the acrylic acid,
wherein
the at least one molecular organic active compound comprises:
no heteroatom other than carbon and hydrogen over and above nitrogen and oxygen,
no nitrogen covalently bonded to one or more hydrogen atoms,
at most one oxygen atom covalently bonded to a hydrogen atom,
no oxygen atom which comprises a covalent double bond to any of the three different carbon atoms, and
neither a radical of an aromatic hydrocarbon nor a radical of a substituted aromatic hydrocarbon, and
the at least one molecular organic active compound has a boiling point of at least 150° C. and at most 350° C. at a pressure of 1.0133·10⁵ Pa, and
a melting point of at most 70° C. at a pressure of 1.0133·10⁵ Pa.

2. The process according to claim 1, wherein the at least one molecular organic active compound comprises more than one tertiary nitrogen atom covalently bonded to each of the three different carbon atoms, with the proviso that none of the three different carbon atoms simultaneously comprises a covalent double bond to any oxygen atom.

3. The process according to claim 2, wherein the at least one molecular organic active compound comprises at least two tertiary nitrogen atoms covalently bonded to each of the three different carbon atoms, with the proviso that none of the three different carbon atoms simultaneously comprises a covalent double bond to any oxygen atom.

4. The process according to claim 2, wherein the at least one molecular organic active compound comprises at least three tertiary nitrogen atoms covalently bonded to each of the three different carbon atoms, with the proviso that none of the three different carbon atoms simultaneously comprises a covalent double bond to any oxygen atom.

5. The process according to claim 1, wherein the at least one molecular organic active compound comprises only tertiary nitrogen atoms covalently bonded to each of the three different carbon atoms, with the proviso that none of the three different carbon atoms simultaneously comprises a covalent double bond to any oxygen atom.

6. The process according to claim 1, wherein the at least one molecular organic active compound comprises no oxygen atom covalently bonded to a hydrogen atom.

7. The process according to claim 1, wherein the at least one molecular organic active compound has a boiling point of at least 160° C. at a pressure of 1.0133·10⁵ Pa.

8. The process according to claim 1, wherein the at least one molecular organic active compound has a boiling point of at most 345° C. at a pressure of 1.0133·10⁵ Pa.

9. The process according to claim 1, wherein the at least one molecular organic active compound has a melting point of at most 60° C. at a pressure of 1.0133·10⁵ Pa.

10. The process according to claim 1, wherein the at least one molecular organic active compound has a melting point of at most −15° C. at a pressure of 1.0133·10⁵ Pa.

11. The process according to claim 1, wherein the at least one molecular active compound is selected from the group consisting of pentamethyldiethylenetriamine, N,N,N′,N′-tetramethyl-1,6-hexanediamine, bis(2-dimethylaminoethyl)ether, 2,2′-dimorpholinodiethyl ether, N,N′-diethylethanolamine, N,N-dimethylcyclohexylamine, N-methylimidazole and 1,2-dimethylimidazole.

12. The process according to claim 1, wherein said thermolyzing, based on a weight of the poly-3-hydroxypropionate, is catalytically effected by 0.01 to 15% by weight of the at least one molecular organic active compound.

13. The process according to claim 1, wherein said thermolyzing, based on a weight of the poly-3-hydroxypropionate, is effected catalytically by up to 50% by weight of the at least one molecular organic active compound.

14. The process according to claim 1, wherein said thermolyzing, based on a weight of the poly-3-hydroxypropionate, is effected catalytically by up to 500% by weight of the at least one molecular organic active compound.

15. The process according to claim 1, wherein said thermolyzing of the poly-3-hydroxypropionate is effected from a solid substance thereof, a melt thereof, a solution thereof in an organic liquid as a solvent, a suspension thereof in the organic liquid as a dispersant, an emulsion thereof in the organic liquid as a dispersant, a biomass thereof, or a slurry of the biomass in an organic solvent as a slurrying agent.

16. The process according to claim 15, wherein the organic liquid has a boiling point of at least 20° C. above a boiling temperature of acrylic acid at a pressure of 1.0133·10⁵ Pa.

17. The process according to claim 15, wherein the organic liquid is selected from the group consisting of an ionic liquid, an oligomeric Michael adduct of acrylic acid onto itself and an addition product, dimethyl sulfoxide, N-methyl-2-pyrrolidone, dialkylformamide, a long-chain paraffinic hydrocarbon, a long-chain alkanol, γ-butyrolactone, ethylene carbonate, diphenyl ether, diglyme, triglyme, tetraglyme, biphenyl, tricresyl phosphate, dimethyl phthalate, diethyl phthalate.

18. The process according to claim 15, wherein a proportion by weight of the poly-3-hydroxypropionate in the solution, the suspension, the emulsion, the biomass, or the slurry of the biomass, is at least 5 to at most 95%.

19. The process according to claim 15, wherein the at least one molecular organic active compound is dissolved in the melt of the poly-3-hydroxypropionate or in the organic liquid.

20. The process according to claim 1, wherein said thermolyzing occurs at a temperature of 50 to 400° C.

21. The process according to claim 1, wherein the process is performed at, above or below an atmospheric pressure.

22. The process according to claim 1, wherein the acrylic acid is discharged continuously with an aid of a stripping gas.

23. The process according to claim 1, wherein said thermolyzing is effected in the presence of at least one polymerization inhibitor.

24. The process according to claim 1, wherein the poly-3-hydroxypropionate is at least one macromolecular compound comprising a structural section of formula (I), 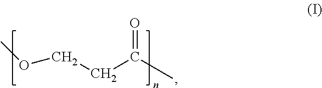
wherein n is an integer ≧6.

25. The process according to claim 24, wherein n≦30 000.

26. The process according to claim 1, wherein the poly-3-hydroxypropionate is a copolymer or a homopolymer.

27. The process according to claim 24, wherein a proportion by weight of the structural section of formula (I) in the poly-3-hydroxypropionate is ≧40%.

28. The process according to claim 1, wherein the poly-3-hydroxypropionate is obtained by a dehydrating polycondensation of 3-hydroxypropionic acid, a ring-opening polymerization of β-propiolactone, a carbonylating reaction of ethylene oxide dissolved in a solvent with CO in the presence of at least one cobalt-comprising catalyst, or a biotechnological means in a biological organism.

29. The process according to claim 1, wherein the poly-3-hydroxypropionate has a polydispersity of ≦2.5.

30. The process according to claim 1, wherein the poly-3-hydroxypropionate has a weight average molecular weight M_{w} of from 1000 to 2 000 000.

31. The process according to claim 1, wherein the poly-3-hydroxypropionate comprises no vinylic head or end group.

32. The process according to claim 1, wherein the acrylic acid is the obtained by converting acrylic acid-comprising gas phase formed in said thermolyzing to a liquid phase by absorption, condensation, or both.

33. The process according to claim 32, wherein
the acrylic acid is separated from the liquid phase in an elevated purity compared to the liquid phase using at least one thermal separation process, and
the at least one thermal separation process comprises a rectification, a crystallization, or both, of the acrylic acid in the liquid phase.

34. The process according to claim 1, further comprising:
subsequently polymerizing the acrylic acid via a free-radical polymerization, optionally in the presence of a conjugate Brønsted base, and optionally in a mixture comprising other mono- or polyunsaturated compounds.

35. The process according to claim 1, wherein the poly-3-hydroxypropionate has a melting point of at most 200° C. at a pressure of 1.0133·10⁵ Pa.

36. The process according to claim 1, wherein the poly-3-hydroxypropionate has a melting point of at least 50° C. at a pressure of 1.0133·10⁵ Pa.

37. The process according to claim 1, wherein the at least one molecular organic active compound comprises no aromatic ring.
